(19) United States
(12) Patent Application Publication
(10) Pub. No.: US 2007/0259344 A1
(43) Pub. Date: Nov. 8, 2007

(54) Compound probes and methods of increasing the effective probe densities of arrays

(75) Inventors: Eric M. Leproust, San Jose, CA (US); Douglas A. Amorese, Los Altos, CA (US); Michael P. Caren, Palo Alto, CA (US); David B. Gordon, Somerville, MA (US); Andrew Payne, Lincoln, MA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(73) Assignee: Agilent Technologies, Inc., Loveland, CO (US)

(21) Appl. No.: 11/417,313

(22) Filed: May 3, 2006

(51) Int. Cl. C12Q 1/68; C07H 21/04; C12M 3/00

(52) U.S. Cl. 435/6; 435/287.2; 536/24.3

(57) ABSTRACT

Methods and articles for analyzing nucleotide sequences of nucleic acid molecules, e.g., using multiple probes per spot of an array, are described. In some embodiments, the methods and articles can reduce the numbers of arrays necessary to probe regions of interest in a biological sample, and/or increase the resolution at which biological events are probed. In some cases, these methods exploit the vertical aspect of an array in order to decrease the number of arrays or spots required for an assay. These probes may be in the form of compound probes, which comprise at least first and second probes, including first and second nucleotide sequences capable of hybridizing to first and second target nucleotide sequences, respectively, in a nucleic acid molecule of interest.

### BACKGROUND

Arrays of nucleic acids have become an increasingly important tool in the biotechnology industry and related fields. These nucleic acid arrays, in which a plurality of distinct or different nucleic acids are positioned on a solid support surface in the form of an array or pattern, find use in a variety of applications, including gene expression analysis, nucleic acid synthesis, drug screening, nucleic acid sequencing, mutation analysis, array CGH, location analysis (also known as ChIP-Chip), and the like.

Arrays having a large number of spots are advantageous in that large genomes or transcriptomes can be assayed at higher resolutions and/or with fewer number of slides per experiment. Current methods of increasing the density of spots per array include forming spots with smaller surface areas and/or positioning spots closer together on the array. Although these methods may be useful, other methods of increasing the effective probe density of arrays would be beneficial.

### SUMMARY OF THE INVENTION

Methods and articles for analyzing nucleotide sequences of nucleic acid molecules are provided.

In one embodiment, an oligonucleotide probe is provided. The oligonucleotide probe comprises a plurality of hybridizing segments, wherein said hybridizing segments hybridize to non-contiguous regions in a target genome.

In some cases, the oligonucleotide probe is at least 60 bases, at least 80 bases, or at least 100 bases in length, up to about 200 or more bases in length. The hybridizing segments may each be, for example, at least 20 bases, at least 30 bases, or at least 40 bases, up to about 50 bases or 60 bases or more in length.

The oligonucleotide probe may comprise at least 2, at least 3, at least 4, at least 5, or at least 6 hybridizing segments. In one embodiment, the hybridizing segments are contiguous. In another embodiment, the hybridizing segments are separated by a linker. The linker may comprise a nucleotide sequence that is optimized to minimize hybridization to the linker.

In some cases, the non-contiguous regions are separated by at least 10 kb in a target mammalian genome. In one particular embodiment, the non-contiguous regions are present on different chromosomes of said mammalian genome.

The invention also provides an array comprising an oligonucleotide probe as described above. In another embodiment, a kit comprising said array is provided.

In another embodiment, a method of sample analysis is provided. The method comprises a) contacting a sample comprising nucleic acids with an array of claim 10 under hybridizing conditions, and b) assessing binding of said nucleic acids with said oligonucleotide.

In one embodiment, a compound probe is provided. The compound probe comprises at least a first oligonucleotide probe comprising a first nucleotide sequence capable of hybridizing to a first target nucleotide sequence in a nucleic acid molecule of interest, and at least a second oligonucleotide probe comprising a second nucleotide sequence capable of hybridizing to a second target nucleotide sequence in the nucleic acid molecule of interest, wherein the first and second nucleotide sequences of the first and second oligonucleotide probes, respectively, together are not genomically contiguous when hybridized to any single strand in the nucleic acid molecule of interest.

In some cases, the first and second nucleotide sequences of the first and second oligonucleotide probes, respectively, are contiguous with each other on the compound probe. In other cases, the first and second nucleotide sequences of the first and second oligonucleotide probes, respectively, are separated from each other by a linker segment on the compound probe, and wherein the first and second nucleotide sequences including the linker segment, together are not genomically contiguous when hybridized to any single strand in the nucleic acid molecule of interest.

In another embodiment, the invention provides a compound probe. The compound probe comprises at least a first oligonucleotide probe comprising a first nucleotide sequence capable of hybridizing to a first target nucleotide sequence in a nucleic acid molecule of interest, and at least a second oligonucleotide probe comprising a second nucleotide sequence capable of hybridizing to a second target nucleotide sequence in the nucleic acid molecule of interest. The first and second nucleotide sequences of the first and second oligonucleotide probes, respectively, may be contiguous with each other on the compound probe or separated from each other by a linker segment on the compound probe, and wherein the first and second nucleotide sequences or first and second nucleotide sequences including the linker segment, together are not genomically contiguous when hybridized to any single strand in the nucleic acid molecule of interest.

In one embodiment, the first and second oligonucleotide probes of a compound probe are contiguous on the compound probe. In another embodiment, the first and second oligonucleotide probes are not contiguous on the compound probe. The first and second nucleotide sequences of the first and second oligonucleotide probes, respectively, may be separated by at least 5 bases if hybridized to a single strand in the nucleic acid molecule of interest, in some embodiments.

The compound probe may have a length of greater than 60, 80, 100, 120, 140, 160, or 180 bases, and, in certain embodiments, may be as large as 200, 250 or 300 bases.

In some instances, the first and second nucleotide sequences of a compound probe are separated by a linker segment. For instance, the compound probe is formed such that a boundary region created by the first and second nucleotide sequences with the linker segment produces less noise than a boundary region created by the first and second nucleotide sequences without the linker segment when hybridized to target nucleotides sequences of a biological sample.

In one embodiment, the first and second nucleotide sequences of the compound probe are not genomic neighbors in the nucleic acid molecule of interest. In another embodiment, the first and second nucleotide sequences are genomic neighbors in the nucleic acid molecule of interest. E.g., the first and second nucleotide sequences may be separated by greater than 1,000 (or greater than 2,000, or greater than 5,000) bases if hybridized to a nucleic acid molecule of interest.

The compound probe can comprise greater than or equal to 2, greater than or equal to 3, greater than or equal to 4, greater than or equal to 5, greater than or equal to 6, or greater than or equal to 8 oligonucleotide probes, in some embodiments.

The invention also provides for an array or array set comprising a plurality of compound probes as described above.

In another embodiment, the invention provides an array or array set for determining a location of a biological phenomenon in terms of chromosomal coordinates in a nucleic acid molecule of interest. The array or array set comprises a plurality of spots, each spot comprising a homogenous composition of nucleotide sequences, each composition of a spot comprising at least a first oligonucleotide probe comprising a first nucleotide sequence capable of hybridizing to a first target nucleotide sequence in a nucleic acid molecule of interest, and at least a second oligonucleotide probe comprising a second nucleotide sequence capable of hybridizing to a second target nucleotide sequence in the nucleic acid molecule of interest. The first and second nucleotide sequences of the first and second oligonucleotide probes, respectively, may be contiguous with each other on the compound probe or separated from each other by a linker segment on the compound probe, and wherein the first and second nucleotide sequences or first and second nucleotide sequences including the linker segment, together are not genomically contiguous when hybridized to any single strand in the nucleic acid molecule of interest.

In some embodiments, the first and second oligonucleotide probes of an array or array set form a compound probe. In one particular embodiment, the first and second oligonucleotide probes are attached by a covalent bond. In some cases, at least two different spots of the array or array set comprise the same oligonucleotide probe. However, sometimes no two spots of the array or array set comprise the same oligonucleotide probe.

In some instances, the first and second target nucleotide sequences are not located on, or near, the same gene in the nucleic acid molecule of interest. The first and second target nucleotide sequences may be separated by greater than 1,000 (or greater than 2,000, or greater than 5,000) bases in the nucleic acid molecule of interest.

In one embodiment, the array or array set comprises at least two spots comprising the first oligonucleotide probe and at least two spots comprising the second of oligonucleotide probe, wherein the array or array set includes a first spot comprising the first and second oligonucleotide probes, and a second spot comprising the first, but not the second, oligonucleotide probe. However, in another embodiment, no two spots of the array or array set comprise the same oligonucleotide probe.

An array or array set of the invention can comprise both regular and compound probes. In some cases, a plurality of spots of an array or array set comprise a homogeneous composition of compounds probes, each compound probe comprising at least 3 probes.

In another embodiment, the invention provides a kit for determining a location of a biological phenomenon in terms of chromosomal coordinates in a nucleic acid molecule of interest. The kit comprises an array or array set comprising a plurality of spots, each spot comprising a homogenous composition of nucleotide sequences, each composition of a spot comprising at least a first oligonucleotide probe comprising a first nucleotide sequence capable of hybridizing to a first target nucleotide sequence in a nucleic acid molecule of interest, and at least a second oligonucleotide probe comprising a second nucleotide sequence capable of hybridizing to a second target nucleotide sequence in the nucleic acid molecule of interest. The first and second nucleotide sequences of the first and second oligonucleotide probes, respectively, may be contiguous with each other on the compound probe or separated from each other by a linker segment on the compound probe, and wherein the first and second nucleotide sequences or first and second nucleotide sequences including the linker segment, together are not genomically contiguous when hybridized to any single strand in the nucleic acid molecule of interest.

In another embodiment, the invention provides a method of determining a location of a biological phenomenon in terms of chromosomal coordinates in a nucleic acid molecule of interest. The method comprises providing an array or array set comprising a plurality of oligonucleotide probes, providing a sample including target nucleotide sequences, contacting the sample with the oligonucleotide probes under conditions that permit hybridization between target nucleotide sequences of the sample and sequences of the oligonucleotide probes, and allowing hybridization of a target nucleotide sequence of the sample and a sequence of the oligonucleotide probe, detecting a signal on the array or array set as a result of hybridization, correlating the signal to at least two locations on the nucleic acid molecule of interest, and determining a location of a biological phenomenon in the nucleic acid molecule of interest.

In some embodiments, a method of determining a location of a biological phenomenon in terms of chromosomal coordinates in a nucleic acid molecule of interest includes providing an array or array set that comprises a plurality of compound probes.

In some cases, determining the location of a biological phenomenon in the nucleic acid molecule of interest comprises comparing a series of signals to an expected distribution of signals. In one embodiment, the biological phenomenon includes binding of protein to the nucleic acid molecule of interest. In another embodiment, the biological phenomenon includes binding of transcription factor to the nucleic acid molecule of interest.

In another embodiment, the invention provides a method of determining a location of a biological phenomenon in terms of chromosomal coordinates in a nucleic acid molecule of interest. The method comprises providing an array or array set comprising a plurality of oligonucleotide probes, providing a sample including target nucleotide sequences, contacting the sample with the oligonucleotide probes under conditions that permit hybridization between target nucleotide sequences of the sample and sequences of the oligonucleotide probes, and allowing hybridization of a target nucleotide sequence of the sample and a sequence of the oligonucleotide probe, producing a signal on the array or array set as a result of hybridization, wherein the signal of a spot alone does not enable determination of the target nucleotide sequence hybridized on the spot, detecting hybridization, and determining a location of a biological phenomenon in the nucleic acid molecule of interest using a combination of signals produced after hybridization.

In another embodiment, the invention provides a method of assaying nucleotide sequences in a biological sample. The method comprises providing a first array or array set comprising a plurality of compound probes, each compound probe comprising at least a first probe including a first nucleotide sequence complementary to a first nucleotide sequence in a biological sample and at least a second probe comprising a second nucleotide sequence complementary to a second nucleotide sequence in the biological sample, contacting a biological sample including target nucleotide sequences with the plurality of compound probes of the first array or array set under conditions that permit hybridization of complementary sequences between the target nucleotide sequences of the biological sample and nucleotide sequences of the first array or array set, detecting hybridized compound probes of the first array or array set, wherein the hybridized compound probes can be hybridized partially or completely, providing a second array or array set comprising a plurality of probes including the first and second probes of the hybridized compound probes of the first array or array set, contacting the biological sample including target nucleotide sequences to the plurality of probes of the second array or array set under conditions that permit hybridization of complementary sequences between the target nucleotide sequences of the biological sample and nucleotide sequences of the second array or array set, and detecting hybridized probes of the second array or array set. In some cases, the second array or array set comprises probes of the compound probes of the first array or array set that gave the strongest signals in the first array or array set, wherein each probe of the second array or array set is presented on a separate spot.

In other words, in certain embodiments, a first array containing compound probes may be contacted with a sample to produce signal producing compound probes. Analysis of the signal-producing probes can indicate which hybridizing segment of the compound probes hybridized to nucleic acids in the sample. In order to confirm that a hybridizing segment of the compound probe hybridized to a nucleic acid in the sample, a second array containing oligonucleotides that contain a single hybridizing segment may be employed. In certain embodiments, the second array may contain the oligonucleotides containing single hybridizing segments, where the hybridizing segments of the oligonucleotides of the second array were identified using the first array. The single hybridizing segments may be present in oligonucleotides that are on different features (i.e., spots) of the second array. In certain embodiments, the second array may contain a higher density of probes for a genomic region of interest than the first array, where the genomic region of interest was identified as being a region of a genome that bound the signal producing probes. As such, in certain methods, the second array may be employed to identify a genomic region to a higher resolution than that possible using the first array.

In another embodiment, the invention provides a compound probe. The compound probe comprises at least a first oligonucleotide probe comprising a first nucleotide sequence capable of hybridizing to a first target nucleotide sequence in a nucleic acid molecule of interest, at least a second oligonucleotide probe comprising a second nucleotide sequence capable of hybridizing to a second target nucleotide sequence in the nucleic acid molecule of interest, and an oligonucleotide linker segment linking the first oligonucleotide probe to the second oligonucleotide probe, and separating the probes from each other, wherein the linker segment is selected to minimize homology noise associated with hybridization of the first nucleotide sequence of the first oligonucleotide probe to the first target nucleotide sequence, and hybridization of the second nucleotide sequence of the second oligonucleotide probe to the second target nucleotide sequence.

In some embodiments, the first and second oligonucleotide probes of the compound probe are contiguous on the compound probe. The compound probe may have a length of greater than 60 bases, greater than 80 bases, or greater than 100 bases. In some instances, the first and second nucleotide sequences of the compound probe are each at least 20 bases in length, at least 30 bases in length, or at least 40 bases in length. In certain embodiments, the first and second nucleotide sequences are not genomic neighbors in the nucleic acid molecule of interest. The compound probe may comprise, for example, greater than or equal to 2, greater than or equal to 3, greater than or equal to 4, greater than or equal to 5, or greater than or equal to 6 oligonucleotide probes. In another embodiment, the invention provides a method of designing a compound probe. The method comprises selecting candidate probes for a compound probe, the candidate probes comprising at least a first oligonucleotide probe comprising a first nucleotide sequence capable of hybridizing to a first target nucleotide sequence in a nucleic acid molecule of interest, and at least a second oligonucleotide probe comprising a second nucleotide sequence capable of hybridizing to a second target nucleotide sequence in the nucleic acid molecule of interest, estimating the boundary homology noise of at least two possible arrangements of the first and second oligonucleotide probes within a compound probe, and selecting the arrangement estimated to have the overall lowest boundary homology noise.

In some embodiments, the first and second nucleotide sequences of the first and second oligonucleotide probes, respectively, are not contiguous in the nucleic acid molecule of interest. For instance, the first and second nucleotide sequences can be separated by at least 5 bases, at least 100 bases, at least 1 kb, or at least 10 kb in the nucleic acid molecule of interest. In some cases, the first and second nucleotide sequences of the first and second oligonucleotide probes, respectively, are present on different chromosomes of a mammalian genome.

In another embodiment, a method comprises estimating the boundary homology noise of all possible arrangements of the first and second oligonucleotide probes within a compound probe, and selecting the arrangement estimated to have the overall lowest boundary homology noise. The method can further comprise selecting a linker segment from a database of linker segments, estimating the boundary homology noise of at least two possible arrangements of the first and second oligonucleotide probes together with the linker segment within a compound probe, and selecting the arrangement estimated to have the overall lowest boundary homology noise. The method can also comprise estimating the boundary homology noise of all possible arrangements of the first and second oligonucleotide probes together with the linker segment within a compound probe, and selecting the arrangement estimated to have the overall lowest boundary homology noise. In some embodiments, the database of linker segments is derived at least in part by sections of the nucleic acid molecule of interest that are known to have good homology scores. In other embodiments, the database of linker segments is derived at least in part by sections of a genome that is different from that of the nucleic acid molecule of interest. The invention also provides for a compound probe, an array or array set, and a kit designed by the process of one or more of the methods described above.

In another embodiment, an array or array set for determining a location of a biological phenomenon in terms of chromosomal coordinates in a nucleic acid molecule of interest is provided. The array or array set comprises at least a first oligonucleotide probe comprising a first nucleotide sequence capable of hybridizing to a first target nucleotide sequence in a nucleic acid molecule of interest, at least a second oligonucleotide probe comprising a second nucleotide sequence capable of hybridizing to a second target nucleotide sequence in the nucleic acid molecule of interest, and an oligonucleotide linker segment linking the first oligonucleotide probe to the second oligonucleotide probe, and separating the probes from each other, wherein the linker segment is selected to minimize homology noise associated with hybridization of the first nucleotide sequence of the first oligonucleotide probe to the first target nucleotide sequence, and hybridization of the second nucleotide sequence of the second oligonucleotide probe to the second target nucleotide sequence.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A and 1B are schematic diagrams of first and second oligonucleotide probes including first and second nucleotide sequences, respectively, attached to an array surface (prior art);

FIG. 1C is a schematic diagram of a microarray including a plurality of spots comprising the oligonucleotide probes of FIGS. 1A and 1B (prior art);

FIGS. 2A-2F are schematic diagrams of different compound probes according to one embodiment of the invention;

FIGS. 3A-3F are schematic diagrams of oligonucleotide probes hybridized to target nucleotides sequences in a nucleic acid molecule of interest according to another embodiment of the invention;

FIG. 4 is a schematic diagram of a microarray including a plurality of spots comprising compound probes according to another embodiment of the invention;

FIGS. 5A and 5B are first and second oligonucleotide probes that may be used in the microarray of FIG. 5C according to another embodiment of the invention;

FIG. 5C is a schematic diagram of a microarray including a plurality of spots comprising multiple probes, which may be used to deconvolute signals produced from the microarray of FIG. 4 according to another embodiment of the invention;

FIG. 6A is a schematic diagram of a microarray including a plurality of spots comprising compound probes according to another embodiment of the invention;

FIG. 6B shows deconvolution of signals produced from the microarray of FIG. 6A according to another embodiment of the invention;

FIG. 7 is a schematic diagram of another microarray including a plurality of spots comprising compound probes according to another embodiment of the invention;

FIGS. 8A and 8B show deconvolution of signals produced after hybridization in the microarray of FIG. 1C;

FIGS. 8C and 8D show data illustrating fitting of intensities to the shape of an expected distribution according to another embodiment of the invention;

FIGS. 9A-9D are deconvolution of signals produced after hybridization in the microarray of FIG. 7 according to another embodiment of the invention;

FIG. 9E is a schematic diagram of signals produced after hybridization in the microarray of FIG. 7, in relation to determining the location of a biological phenomenon in terms of chromosomal coordinates in a nucleic acid molecule of interest, according to another embodiment of the invention;

FIGS. 10A and 10B show signals (log-ratio enrichments) produced after hybridization in the microarray of FIG. 7, and how a single compound probe can provide information associated with multiple chromosomal locations according to another embodiment of the invention; and

FIGS. 11A, 11B, and 11C show signals (log-ratio enrichments) after hybridization of compound probes.

### DEFINITIONS

Unless defined otherwise, all technical and scientific terms used herein have the same meaning as commonly understood by one of ordinary skill in the art to which this invention belongs. Certain elements are defined below for the sake of clarity and ease of reference.

A “biopolymer” is a polymeric biomolecule of one or more types of repeating units. Biopolymers are typically found in biological systems and particularly include polysaccharides (e.g., carbohydrates), peptides (which term is used to include polypeptides and proteins) and oligonucleotides, as well as their analogs such as those compounds composed of or containing amino acid analogs or non-amino acid groups, or nucleotide analogs or non-nucleotide groups.

The terms “ribonucleic acid” and “RNA” as used herein refer to a polymer composed of ribonucleotides.

The terms “deoxyribonucleic acid” and “DNA” as used herein mean a polymer composed of deoxyribonucleotides.

The term “mRNA” means messenger RNA.

The term “biomolecule” means any organic or biochemical molecule, group or species of interest that may be formed in an array on a substrate surface. Exemplary biomolecules include peptides, proteins, amino acids and nucleic acids.

The term “peptide” as used herein refers to any compound produced by amide formation between a carboxyl group of one amino acid and an amino group of another group.

The term “oligopeptide” as used herein refers to peptides with fewer than about 10 to 20 residues, i.e., amino acid monomeric units.

The term “polypeptide” as used herein refers to peptides with more than 10 to 20 residues.

The term “protein” as used herein refers to polypeptides of specific sequence of more than about 50 residues.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “polynucleotide” or “nucleic acid” refers to a polymer composed of nucleotides, natural compounds such as deoxyribonucleotides or ribonucleotides, or compounds produced synthetically (e.g., PNA as described in U.S. Pat. No. 5,948,902 and the references cited therein), which can hybridize with naturally-occurring nucleic acids in a sequence specific manner analogous to that of two naturally occurring nucleic acids, e.g., can participate in Watson-Crick base pairing interactions. The polynucleotide can have from about 20 to 5,000,000 or more nucleotides. The larger polynucleotides are generally found in the natural state. In an isolated state the polynucleotide can have about 30 to 50,000 or more nucleotides, usually about 100 to 20,000 nucleotides, more frequently 500 to 10,000 nucleotides. Isolation of a polynucleotide from the natural state often results in fragmentation. It may be useful to fragment longer target nucleic acid sequences, particularly RNA, prior to hybridization to reduce competing intramolecular structures.

The polynucleotides include nucleic acids, and fragments thereof, from any source in purified or unpurified form including DNA (dsDNA and ssDNA) and RNA, including tRNA, mRNA, rRNA, mitochondrial DNA and RNA, chloroplast DNA and RNA, DNA/RNA hybrids, or mixtures thereof, genes, chromosomes, plasmids, cosmids, the genomes of biological material such as microorganisms, e.g., bacteria, yeasts, phage, chromosomes, viruses, viroids, molds, fungi, plants, animals, humans, and the like. The polynucleotide can be only a minor fraction of a complex mixture such as a biological sample. Also included are genes, such as hemoglobin gene for sickle-cell anemia, cystic fibrosis gene, oncogenes, cDNA, and the like.

The polynucleotide can be obtained from various biological materials by procedures well known in the art. The polynucleotide, where appropriate, may be cleaved to obtain a fragment that contains a target nucleotide sequence, for example, by shearing or by treatment with a restriction endonuclease or other site-specific chemical cleavage method.

For purposes of this invention, the polynucleotide, or a cleaved fragment obtained from the polynucleotide, will usually be at least partially denatured or single stranded or treated to render it denatured or single stranded. Such treatments are well known in the art and include, for instance, heat or alkali treatment, or enzymatic digestion of one strand. For example, dsDNA can be heated at 90 to 100 degrees Celcius for a period of about 1 to 10 minutes to produce denatured material.

The nucleic acids may be generated by in vitro replication and/or amplification methods such as the Polymerase Chain Reaction (PCR), asymmetric PCR, the Ligase Chain Reaction (LCR) and so forth. The nucleic acids may be either single-stranded or double-stranded. Single-stranded nucleic acids are preferred because they lack complementary strands that compete for the oligonucleotide precursors during the hybridization step of the method of the invention.

The term “oligonucleotide” refers to a polynucleotide, usually single stranded, usually a synthetic polynucleotide but may be a naturally occurring polynucleotide. The length of an oligonucleotide is generally governed by the particular role thereof, such as, for example, probes (e.g., compound probes), primers, X-mers, and the like. Various techniques can be employed for preparing an oligonucleotide. Such oligonucleotides can be obtained by biological synthesis or by chemical synthesis. For short oligonucleotides (i.e., up to about 100 nucleotides), chemical synthesis will frequently be more economical as compared to the biological synthesis. In addition to economy, chemical synthesis provides a convenient way of incorporating low molecular weight compounds and/or modified bases during specific synthesis steps. Furthermore, chemical synthesis is very flexible in the choice of length and region of the target polynucleotide binding sequence. The oligonucleotide can be synthesized by standard methods such as those used in commercial automated nucleic acid synthesizers. Chemical synthesis of DNA on a suitably modified glass or resin can result in DNA covalently attached to the surface. This may offer advantages in washing and sample handling. Methods of oligonucleotide synthesis include phosphotriester and phosphodiester methods (Narang, et al. (1979) Meth. Enzymol 68:90) and synthesis on a support (Beaucage, et al. (1981) Tetrahedron Letters 22:1859-1862) as well as phosphoramidite techniques (Caruthers, M. H., et al., “Methods in Enzymology,” Vol. 154, pp. 287-314 (1988)) and others described in “Synthesis and Applications of DNA and RNA,” S. A. Narang, editor, Academic Press, New York, 1987, and the references contained therein. The chemical synthesis via a photolithographic method of spatially addressable arrays of oligonucleotides bound to glass surfaces is described by A. C. Pease, et al. (Proc. Nat. Acad. Sci. USA 91:5022-5026, 1994). In some cases, synthesis of certain oligonucleotides (e.g., compound probes) can be performed according to methods disclosed in U.S. Patent Publication No. 2005/0214779, filed Mar. 29, 2004, entitled “Methods for in situ generation of nucleic acid arrays”, which is incorporated herein by reference.

Generally, as used herein, the terms “oligonucleotide” and “polynucleotide” are used interchangeably. Further, generally, the term “nucleic acid molecule” also encompasses oligonucleotides and polynucleotides.

The term “oligonucleotide” refers to a nucleic acid molecule that contains at least 3 nucleotides, in some cases, 4 to 14 nucleotides, in other cases 5 to 20, 5 to 30, 8 to 50, 8 to 60, 50 to 100, 50 to 120, 50 to 150, 100-200 nucleotides in length, or longer. An oligonucleotide of a certain length X may be referred to as an X-mer. For instance, a 60-mer refers to an oligonucleotide having a sequence of 60 nucleotides.

The term “X-mer precursors”, sometimes referred to as “oligonucleotide precursors” refers to a nucleic acid sequence that is complementary to a portion of the target nucleic acid sequence. The oligonucleotide precursors are sequences of nucleoside monomers joined by phosphorus linkages (e.g., phosphodiester, alkyl and aryl-phosphate, phosphorothioate, phosphotriester), or non-phosphorus linkages (e.g., peptide, sulfamate and others). They may be natural or non-natural (e.g., synthetic) molecules of single-stranded DNA and single-stranded RNA with circular, branched or linear shapes, and optionally including domains capable of forming stable secondary structures (e.g., stem-and-loop and loop-stem-loop structures). The oligonucleotide precursors contain a 3′-end and a 5′-end.

The term “oligonucleotide probe” or “probe” refers to an oligonucleotide employed to hybridize to a portion of a polynucleotide such as another oligonucleotide or a target nucleotide sequence. The design and preparation of the oligonucleotide probes are generally dependent upon the sequence to which they hybridize. Oligonucleotide probes can include natural or non-natural nucleotides.

The phrase “nucleic acid molecule bound to a surface of a solid support” or “probe bound to a solid support” or a “target bound to a solid support” or “polynucleotide bound to a solid support” refers to a nucleic acid molecule (e.g., an oligonucleotide or polynucleotide) or mimetic thereof (e.g., comprising at least one PNA or LNA monomer) that is immobilized on a surface of a solid substrate, where the substrate can have a variety of configurations, e.g., including, but not limited to, planar, non-planar, a sheet, bead, particle, slide, wafer, web, fiber, tube, capillary, microfluidic channel or reservoir, or other structure. In certain embodiments, collections of nucleic acid molecules are present on a surface of the same support, e.g., in the form of an array, which can include at least about two nucleic acid molecules, which may be identical or comprise a different nucleotide base composition. As used herein, the terms “bound to a solid support” and “attached to a solid support” may be used interchangeably unless context dictates otherwise. “Addressable sets of probes” and analogous terms refer to the multiple known regions of different moieties of known characteristics (e.g., base sequence composition) supported by or intended to be supported by a solid support, i.e., such that each location is associated with a moiety of a known characteristic and such that properties of a target moiety can be determined based on the location on the solid support surface to which the target moiety hybridizes under stringent conditions.

A solid support, in some embodiments, is non-porous. In certain embodiments, a non-porous support comprises a bead. As used herein, a “non-porous support” refers to a support having a pore size that essentially excludes synthesis reagents (e.g., such as biopolymer precursors or solutions for preparing biopolymers, including but not limited to deblocking and purging solutions) from entering the support (e.g., penetrating the surface). In one aspect, to the extent there are any openings/pores in a surface of a support, the openings/pores can be less than about 100 Angstroms, less than about 60 angstroms, less than about 50 Angstroms, less than about 25 Angstroms, etc. Included in this definition are supports having these specified size restrictions or properties in their natural state or which have been treated to reduce the size of any openings/pores to obtain these restrictions/properties. In certain embodiments, supports include non-porous beads. Such beads can be fabricated as is known in the art, for example, as described in U.S. Patent Publication No. 2003/0225261.

An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding (or hybridization) agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

An “array set” includes one or more arrays tailored to a particular assay. An array set may include more than one array, e.g., when there are too many spots or features to fit on a single substrate and/or spots are spread over multiple substrates. The multiple substrates may be said to be part of an array set. An example of an array set includes a “10-set” product, which is on ten glass slides with about 440,000 spots (e.g., about 44 k spots per slide). An “array” and “array set” may be used interchangeably herein in some embodiments of the invention.

Any given substrate may carry any number of oligonucleotides on a surface thereof. In one embodiment, one, two, four, or more arrays are disposed on a front surface of the substrate. Depending upon the use, any, or all, of the arrays may be the same or different from one another and each may include multiple spots or features of different moieties (for example, different polynucleotide sequences). A spot or feature of an array is generally homogeneous in composition and in concentration. A region at a particular predetermined location (an “address”) on the array will detect a particular target or set of targets (although a spot or feature may incidentally detect non-targets of that spot or feature). The target for which the spot or feature is specific is, in representative embodiments, known.

A typical array may contain more than ten, more than one hundred, more than one thousand more ten thousand spots, more than one hundred thousand spots, or even more than one million spots in an area of less than 20 cm² or even less than 10 cm². For example, spots may have widths (that is, diameter, for a round spot) in the range from 10 μm to 1.0 cm. In other embodiments, each spot may have a width in the range of 1.0 μm to 1.0 mm, usually 5.0 μm to 500 μm, and more usually 10 μm to 200 μm. Non-round spots may have area ranges equivalent to that of circular spots with the foregoing width (diameter) ranges. At least some, or all, of the spots are of different compositions (for example, when any repeats of each spot composition are excluded, the remaining spots may account for at least 5%, 10%, or 20% of the total number of spots).

In some embodiments, interspot areas will typically (but not essentially) be present which do not carry any oligonucleotide (or other biopolymer or chemical moiety of a type of which the features are composed). Such interspot areas typically will be present where the arrays are formed by processes involving drop deposition of reagents but may not be present when, for example, light directed synthesis fabrication processes are used. It will be appreciated though, that the interfeature areas, when present, could be of various sizes and configurations. In other embodiments, however, oligonucleotides may be present in interspot areas. In one particular embodiment, spots are arranged adjacent one another such that there are no interspot areas between each spot.

Each array may cover an area of less than 100 cm², or even less than 50 cm², 10 cm² or 1 cm². In certain embodiments, the substrate carrying the one or more arrays will be shaped as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either oligonucleotide precursor units (such as monomers) in the case of in situ fabrication, or the previously obtained oligonucleotide. Such methods are described in detail in, for example, the previously cited references including U.S. Pat. Nos. 6,242,266; 6,232,072; 6,180,351; 6,171,797; 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein.

The term “biological sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained, e.g., after culturing and/or one or more processing steps. In one embodiment, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell or cell type. Genomic sequences include, but are not limited to, those involved in the maintenance, replication, segregation, and generation of higher order structures (e.g. folding and compaction of DNA in chromatin and chromosomes), or other functions, if any, of nucleic acids, as well as all the coding regions and their corresponding regulatory elements needed to produce and maintain each virus, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence. In certain aspects, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

The term “target nucleic acid sequence” refers to a sequence of nucleotides to be identified, detected or otherwise analyzed, usually existing within a portion or all of a polynucleotide. In the present invention, the identity of the target nucleotide sequence may or may not be known. The identity of the target nucleotide sequence may be known to an extent sufficient to allow preparation of various sequences hybridizable with the target nucleotide sequence and of oligonucleotides, such as probes and primers, and other molecules necessary for conducting methods in accordance with the present invention and so forth. Determining the sequence of the target nucleic acid includes in its definition, determining the sequence of the target nucleic acid or sequences within regions of the target nucleic acid to determine the sequence de novo, to resequence, and/or to detect mutations and/or polymorphisms. In some cases, target nucleic acid sequences are present in a biological sample of interest.

The terms “target nucleic acid” and “nucleic acid molecule of interest” are used interchangeably herein. A target nucleic acid or a nucleic acid molecule of interest may represent, for example, a genome (e.g., a “target genome”) or a transcriptome (e.g., a “target transcriptome”).

The target sequence may contain from about 30 to 5,000 or more nucleotides, or from 50 to 1,000 nucleotides. In some cases, the target nucleotide sequence is generally a fraction of a larger molecule. In other cases, the target nucleotide sequence may be substantially the entire molecule, such as a polynucleotide as described above. The minimum number of nucleotides in the target nucleotide sequence is selected to assure that the presence of a target polynucleotide in a sample is a specific indicator for the presence of polynucleotide in a sample. The maximum number of nucleotides in the target nucleotide sequence is normally governed by several factors: the length of the polynucleotide from which it is derived, the tendency of such polynucleotide to be broken by shearing or other processes during isolation, the efficiency of any procedures required to prepare the sample for analysis (e.g., transcription of a DNA template into RNA) and the efficiency of identification, detection, amplification, and/or other analysis of the target nucleotide sequence, where appropriate.

The terms “hybridization”, and “hybridizing”, in the context of nucleotide sequences are used interchangeably herein. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the hybridization of the two sequences. Increased stringency can be achieved by elevating the temperature, increasing the ratio of co-solvents, lowering the salt concentration, and the like. Hybridization also includes in its definition the transient hybridization of two complementary sequences. It is understood by those skilled in the art that non-covalent hybridization between two molecules, including nucleic acids, obeys the laws of mass action. Therefore, for purposes of the present invention, hybridization between two nucleotide sequences for a length of time that permits primer extension and/or ligation is within the scope of the invention. The term “hybrid” refers to a double-stranded nucleic acid molecule formed by hydrogen bonding between complementary nucleotides.

The term “complementary, “complement,” or “complementary nucleic acid sequence” refers to the nucleic acid strand that is related to the base sequence in another nucleic acid strand by the Watson-Crick base-pairing rules. In general, two sequences are complementary when the sequence of one can hybridize to the sequence of the other in an anti-parallel sense wherein the 3′-end of each sequence hybridizes to the 5′-end of the other sequence and each A, T(U), G, and C of one sequence is then aligned with a T(U), A, C, and G, respectively, of the other sequence. RNA sequences can also include complementary G/U or U/G basepairs.

In certain embodiments, an array is contacted with a nucleic acid sample under stringent assay conditions, i.e., conditions that are compatible with producing hybridized pairs of biopolymers of sufficient affinity to provide for the desired level of specificity in the assay while being less compatible to the formation of hybridized pairs between members of insufficient affinity. Stringent assay conditions are the summation or combination (totality) of both hybridization conditions and wash conditions for removing unhybridized molecules from the array.

As known in the art, “stringent hybridization conditions” and “stringent hybridization wash conditions” in the context of nucleic acid hybridization are sequence dependent, and are different under different experimental parameters. Stringent hybridization conditions include, but are not limited to, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization in 0.5 M NaHPO4, 7% sodium dodecyl sulfate (SDS), 1 mM EDTA at 65° C., and washing in 0.1×SSC/0.1% SDS at 68° C. can be performed. Additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

Wash conditions used to remove unhybridized nucleic acids may include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature. Other methods of agitation can be used, e.g., shaking, spinning, and the like.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional hybridized complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate. The term “highly stringent hybridization conditions” as used herein refers to conditions that are compatible to produce complexes between complementary members, i.e., between immobilized probes and complementary sample nucleic acids, but which does not result in any substantial complex formation between non-complementary nucleic acids (e.g., any complex formation which cannot be detected by normalizing against background signals to interfeature areas and/or control regions on the array).

Additional hybridization methods are described in references describing CGH techniques (Kallioniemi et al., Science 1992;258:818-821 and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol. 1981;21:470-480 and Angerer et al., In Genetic Engineering: Principles and Methods, Setlow and Hollaender, Eds. Vol. 7, pgs 43-65 (Plenum Press, New York 1985). See also U.S. Pat. Nos. 6,335,167; 6,197,501; 5,830,645; and 5,665,549; the disclosures of which are herein incorporated by reference.

The term “tag” as used herein, generally refers to a chemical moiety, which is used to identify a nucleic acid sequence, and preferably but not necessarily to identify a unique nucleic acid sequence. For instance, “tags” with different molecular weights can be distinguishable by mass spectrometry, and may be used to reduce the mass ambiguity between two or more nucleic acid molecules with different nucleotide sequences, but with the identical molecular weights. The “tag” may be covalently linked to an X-mer precursor, e.g., through a cleavable linker.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

As used herein, “not genomically contiguous” means that the binding sites of a first hybridizing segment of an oligonucleotide (e.g., a first probe of a compound probe) and a second hybridizing segment of the oligonucleotide (e.g., a second probe of a compound probe) are not contiguous in a target genome. Non-genomically contiguous sequences may be separated by at least 5 bases, at least 100 bases, at least 1 kb, at least 10 kb, at least 100 kb and in certain cases may be on different chromosomes in a genome, e.g., a mammalian, e.g., human genome, etc.

A “signal” is a numerical measurement or an estimated (e.g., calculated) measurement of a characteristic of a signal received from scanning an array. Thus, a signal is a numerical score that quantifies some aspect of a spot/spot signal. For example, a mean intensity value of a spot is a statistic, as is a standard deviation value for pixel intensity within a spot. A signal can also refer to the “enrichment” of the probe, including, but not limited to, so-called “one-color” measurements, ratios between channels of a “two-color” assay, difference between channels of a “two-color” assay, or variants of these measures that are adjusted by normalization or by using estimates of the error in the measurements.

As used herein, “enrichment” refers to a signal or a meaningful combination of signals (e.g., of two colors of the same spot). For instance, in some embodiments, the scanner can measure two signal strengths for each feature: (1) the strength of a signal at a first wavelength that indicates the strength of the binding between the probes of a given feature and a control target; and (2) the strength of a signal at a second wavelength that indicates the strength of the binding between the probes of the aforementioned given feature and a test target. The ratio between the two signal strengths indicates the extent by which the test target differs from the control, and may indicate that a particular region of the genome is of interest. Thus, a high ratio between signal strengths from a test target and a control target (test:control) typically indicates a region of interest. The ratio is one of a number of possible ways of measuring the “enrichment” of the test target. Others include so-called “one-color” measurements (test), difference (test-control), or variants of these measures that are adjusted by normalization or by using estimates of the error in the measurements (test-control)/error. In certain embodiments, “signal” and “enrichment” are used interchangeably herein.

A “hybridizing segment” is a region of an oligonucleotide that hybridizes with a target nucleic acid.

As used herein, “homology noise” (or “cross-hybridization noise”) refers to a signal produced by hybridization of a probe to a DNA fragment that do not correspond to the genomic location represented by the probe. This signal can occur, for instance, when DNA fragments from different locations in the genome have sequences similar to all, or a portion of, a probe (e.g., high homology). This signal can also occur in some methods involving formation of compound probes, e.g., when sequences that form the hybridizing segments of the compound probe are concatenated, creating new sequences at the concatenation point.

### DETAILED DESCRIPTION

The present invention relates to methods and apparatus for analyzing nucleotide sequences of nucleic acid molecules and, more specifically, to methods and apparatus for analyzing nucleotide sequences of nucleic acid molecules using multiple probes per spot of an array. The present inventors have developed methods to reduce the numbers of arrays necessary to probe regions of interest in a biological sample and to increase the resolution at which biological events are probed. In some cases, these methods exploit the vertical aspect of an array in order to decrease the number of arrays or spots required for an assay at a given level of information deliverable by the assay. In one embodiment, spots of an array may include long probes (e.g., probes comprising greater than about 60 bases). These probes may be in the form of compound probes, which comprise at least first and second probes, including first and second nucleotide sequences capable of hybridizing to first and second target nucleotide sequences, respectively, in a nucleic acid molecule of interest. As such, a single spot of an array may include several different probes, which can increase the probe density of an array. The design of compound probes, in accordance with the invention—including two or more different probes (i.e., probes having different nucleic acid sequences)—can reduce the number of spots or arrays necessary to query the interactions of a large nucleic acid molecule of interest.

The invention also provides compound probes, including probes designed to minimize or eliminate any complication (e.g., false “hits”) resulting from boundary sequences between probes. That is, compound probes can be defined by individual probes directly attached in sequence, or can include multiple probes at least some of which are separated by non-probe sequences. In either case, boundaries between probes, or between a probe(s) and a boundary sequence, can be taken into account in deconvolution or deciphering of hybridization information to determine ultimate desired information from biological events in an assay. These aspects are described in greater detail below.

Each of the following commonly-owned applications directed to related subject matter and/or disclosing methods and/or devices and/or materials useful or potentially useful for the practice of the present invention is incorporated herein by reference: a U.S. patent application filed on even date herewith, entitled “Methods of Increasing the Effective Probe Densities of Arrays,” by Gordon, et al.; a U.S. patent application filed on even date herewith entitled “Analysis of Arrays,” by Gordon, et al.; and a U.S. patent application filed on even date herewith, entitled “Target Determination using Compound Probes,” by Sampas.

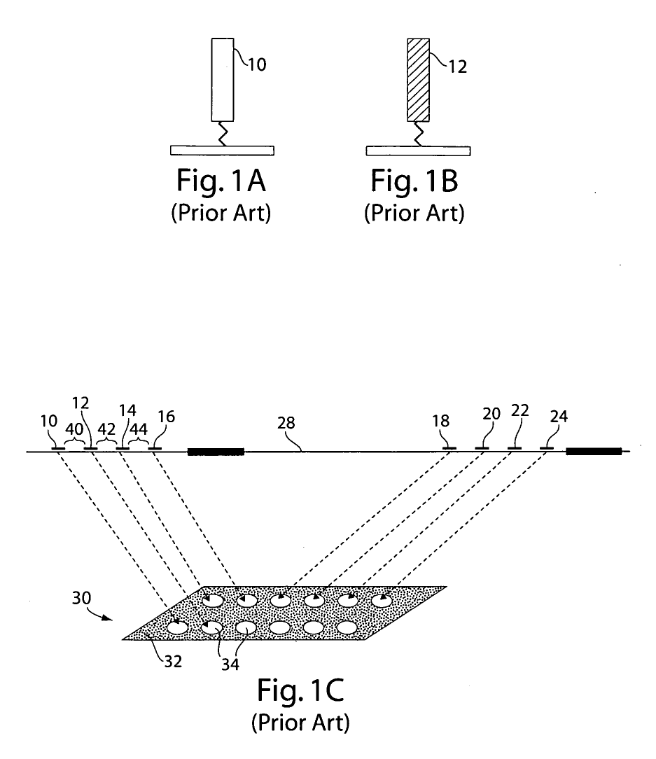

FIGS. 1A and 1B show typical (e.g., regular) probes 10 and 12 that can be designed to hybridize to target nucleotide sequences. The target nucleotide sequences may be a portion of a larger molecule such as a polynucleotide. As such, probes 10 and 12 may have a suitable length such that they can be used to assay nucleotide sequences in a biological sample. Typically, the lengths of probes 10 and 12 are between 8 and 60 nucleotide sequences. For instance, probe 10 may be a 10 mer, 20 mer, 30 mer, 40 mer, 50 mer, or 60 mer.

In the embodiment shown in FIG. 1C, a series of probes 10-24 may be designed to hybridize to nucleotide sequences located on different parts of a nucleic acid molecule of interest 28, which may represent a genome or a transcriptome (e.g., of a mammal). Probes 10-24 may be immobilized on, e.g., covalently attached to, locations on solid support 32 (e.g., a substrate surface) of assay 30. Each distinct probe on the support may be present as a homogeneous composition of multiple copies of the probe on the substrate surface, e.g., as spots or features 34 on the surface of the substrate.

In some embodiments of the invention, methods of reducing the number of arrays used to probe regions of interest in a biological sample and increasing the resolution at which biological events are probed can be achieved by using spots or features comprising a homogeneous composition of multiple probes. Spots including a homogeneous composition of at least first and second probes may involve arranging the first and second probes vertically with respect to each other. For example, the first probe may be positioned on top of the second probe, or the second probe may be positioned on top of the first probe in the spot. In some instances, the first and second probes may be unattached to each other in the spot. For example, the first probe may be attached directly to the surface and the second probe may be printed or synthesized on top of the first probe. Printing may include, in certain instances, chemical attachment of a first probe to a second, and/or the synthesis of one probe on top of a second probe, for instance, one or more bases at a time. The first and second probes may be chemically associated with one another on the spot (e.g., by hydrogen bonding, van der Waals forces, etc.). As such, the height of the probes in each spot can provide another dimension for performing hybridization assays. In another embodiment, a first probe may be positioned on top of a second probe and the first and second probes may be attached (e.g., by a covalent bond) to form a compound probe, as discussed in more detail below. As such, the present inventors have developed a vertically differential array (in addition to horizontally differential array aspects, all in the context of a horizontal assay support surface where used), in order to decrease the number of arrays or spots required in an assay for a given amount of information determinable by the array.

Arrays of the invention can take a variety of forms. For example, an array may include a plurality of spots, each spot comprising a homogeneous composition of nucleotide sequences, each composition of a spot comprising at least a first and a second oligonucleotide probe. The first and second oligonucleotide probes may comprise first and second nucleotide sequences, respectively, capable of hybridizing to a first and second target nucleotide sequence in the nucleic acid molecule of interest. In some cases, the first and second nucleotide sequences of the first and second oligonucleotide probes together are not genomically contiguous when hybridized to any single strand in the nucleic acid molecule of interest. Additionally and/or alternatively, in some embodiments, the first and second nucleotide sequences of the first and second oligonucleotide probes, along with any linker segments that may be present on the first and/or second probes, together are not genomically contiguous when hybridized to any single strand in the nucleic acid molecule of interest, as described in greater detail below. In some embodiments, the first and second probes may be separated by at least 5 bases if hybridized to a single strand in the nucleic acid molecule of interest. In other cases, the first and second nucleotide sequences of the first and second oligonucleotide probes may overlap if hybridized to a single strand in the nucleic acid molecule of interest.

In some embodiments, at least first and second oligonucleotide probes may be printed together to form a single spot of an array (e.g., on top of each other, beside one another, or in a mixture), and the first and second probes may be capable of hybridizing to target nucleic acid sequences in a sample. In this arrangement, the first and second probes might not be chemically attached to each other (e.g., by a covalent bond). For instance, in one embodiment, the first and second probes can be individually and separately immobilized with respect to the array supporting surface. In another embodiment, the first and second probes can be concatenated. In yet another embodiment, the first and second probes can be synthesized off-line, mixed, deposited, and immobilized on the surface. Of course, greater than two probes, e.g., third, fourth, fifth, or sixth probes, can be printed to form a single spot on the array, and the array or array set can comprise a plurality of such spots. In some cases, an array or array set can be fabricated with higher multiples of probes on spots, where the ratio of number of probes per spot can be varied between spots (e.g., 10:30:60 or 30:60:10). Other suitable arrangements of the first, second, and higher numbers of oligonucleotide probes on a spot are also possible, and are contemplated within the scope of the present invention. For instance, some or all of the compound probes can be suspended in a liquid phase mixture, and then attached to a surface during hybridization, e.g., using a specific linker sequence that attaches the compound probes to predetermined sites on the surface of a substrate.

In the examples of the configurations described above, a single spot signal may be read from each spot. In some embodiments, the spot signal is an aggregated signal from all of the signals contributed by each of the probes of the spot, and the signals from each of the probes may be indistinguishable from one another. Deconvolution or decoding of the signals may be required in order to determine, if desired, which probe(s) contributed to the spot signal.

In other embodiments, first and second oligonucleotide probes may be attached to one another as a single probe, forming a compound probe. A compound probe may include at least first and second oligonucleotide probes including first and second nucleotide sequences, respectively, that are contiguous with each other or separated from each other by a linker segment on the compound probe, where the at least first and second nucleotide sequences or first and second nucleotide sequences including the linker segment, together are not genomically contiguous when hybridized to any single strand in the nucleic acid molecule of interest. In some cases, e.g., when the first and second nucleotide sequences of the probes are substantially different, the first and second nucleotide sequences may be separated (e.g., in terms of genomic coordinates) by at least 5 bases if hybridized to a single strand in the nucleic acid molecule of interest. In some embodiments, a compound probe is an oligonucleotide probe comprising a plurality of hybridizing segments, wherein the hybridizing segments hybridize to non-contiguous regions in a target genome.

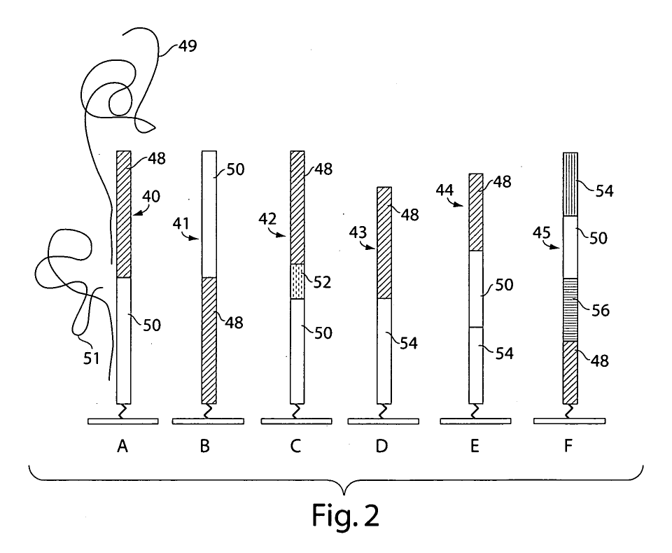

Non-genomically contiguous sequences are spaced by at least 5 bases, at least 100 bases, at least 1 kb, at least 10 kb, at least 100 kb and in certain cases may be on different chromosomes in a genome, e.g., a mammalian, e.g., human genome, etc. Configurations and arrangements of probes within a compound probe may vary, as illustrated in more detail below. Each probe of a compound probe may have a suitable length such that it can be used to hybridize to target nucleotide sequences in a biological sample. As shown in FIG. 2A, compound probe 40 includes at least a first probe 48 and a second probe 50. First probe 48 and second probe 50 may be made up of different nucleic acid sequences and may hybridize to different portions of a nucleic acid molecule of interest, or different nucleic acid molecules. For instance, all, or a portion, of probe 48 may hybridize to a first target nucleotide sequence indicated as strand 49 in the figure, and all, or a portion, of probe 50 may hybridize to a portion of target nucleotide sequence 51.

In other instances, a compound probe may include at least first and second probes that are substantially similar. For instance, all, or portions, of the nucleotide sequences of the first and second probe may comprise the same sequence. E.g., the first and second probes may be designed to hybridize to an essentially identical portion of a nucleic acid molecule of interest. In such a case, the first and second probes may have the same lengths in some embodiments; however, in other embodiments, the first and second probes may have different lengths. A compound probe including first and second probes that are substantially similar may be advantageous for increasing the accuracy of hybridization in an assay.

As compound probes may vary, an array or array set of the invention can include one, or a combination, of types of compound probes described herein. Arrays and array sets of probes and compound probes are described in more detail below. In addition, an array or array set may comprise any combination of both compound probes and typical non-compound (e.g., regular) probes.

As illustrated in FIGS. 2A and 2B, the orientation of probes 48 and 50 may vary on compound probe 40 compared to compound probe 41. Certain designs of compound probes, e.g., orientations of probes on a compound probe and/or ordering of the compound probes within the probe, may be advantageous when considering, for example, decreasing the noise of a signal and/or the ability to synthesize the probe. Design considerations for compound probes are described in more detail below.

FIGS. 2A and 2B show oligonucleotide probes that are contiguous with each other on the compound probe. For instance, the first probe comprising a first nucleotide sequence may be directly adjacent to the second probe comprising a second nucleotide sequence. In other cases, the first and second nucleotide sequences of first and second oligonucleotide probes, respectively, are not contiguous with each other on the compound probe. For example, compound probes may be separated by a linker segment 52, which may comprise specific nucleic acid sequences (FIG. 2C). In some embodiments, for example, the specific nucleic acid sequence of linker segment 52 does not include a sequence that makes probes 48 and 50, along with linker segment 52, genomically contiguous when each of the probes and segments is hybridized to any single strand in the nucleic acid molecule of interest, as discussed in more detail below. As shown in FIG. 2C, probes 48 and 50 may be shorter (i.e., include few nucleotide sequences) if linker segments are included on the compound probe (e.g., compared to the lengths probes 48 and 50 in FIG. 2A and 2B). However, in other instances, e.g., depending on the lengths of the probes and/or the total length of the compound probe, the lengths of probes 48 and 50 may not differ compared to compound probes without linker segments.

In some embodiments, e.g., as illustrated in FIG. 2D, compound probe 43 may include probe 48 and a probe 54 having a “control” sequence. The control sequence may be a “negative control” sequence that is not complementary to any part of the genomic sequence, or it may a “positive control” sequence designed to be complementary to either genomic regions, or to other DNAs added (“spiked-in”) to the biological material at a stage prior to hybridization.

A compound probe may optionally comprise a third probe 54, as shown in compound probe 44 of FIG. 2E, or a fourth probe 56, as shown in compound probe 45 of FIG. 2F. Of course, greater than four probes, e.g., five, six, seven, or higher numbers of probes, can be included on a compound probe. I.e., in some cases, a compound probe can comprise greater than 2, greater than 4, greater than 6, greater than 8, greater than 10, greater than 12, greater than 14, or greater than 16 probes. In certain embodiments, a compound probe can comprise 3, 5, 7, 9, 11, 13, 15, 17, or 20 probes. As noted, at least two probes of a compound probe may have different sequences and may hybridize to a particular portion of the nucleic acid molecule of interest. I.e., greater than 50%, greater than 70%, greater than 90%, or about 100% of the sequences of a first probe may differ from those of a second probe, as described in more detail below.

Compound probes 40-45 of FIG. 2 may have various lengths and/or may comprise various numbers of nucleotides. For instance, a compound probe may comprise greater than or equal to 20 nucleotides, greater than or equal to 40 nucleotides, or greater than or equal to 60 nucleotides. In some cases, compound probe 40 (and/or compound probes 41-45) forms a long, high quality oligonucleotide. E.g., the compound probe may comprise greater than or equal to 80 nucleotides, greater than or equal to 100 nucleotides, greater than or equal to 120 nucleotides, greater than or equal to 140 nucleotides, or greater than or equal to 160 nucleotides. In certain instances, compound probe 40 (and/or compound probes 41-45) may be a 50 mer, 70 mer, 90 mer, 110 mer, 130 mer, 150 mer, or 170 mer. In certain embodiments a probe, i.e., a hybridizing segment of a compound probe, may be in the range of 30 to 80 nt in length, e.g., 30 to 40 nt in length 40 to 50 nt in length, 50 to 60 nt in length or 70 to 80 nt in length.

The first nucleotide sequence (i.e., the first hybridizing segment) within a compound probe that is selected to hybridize at least a portion of the target nucleic acid may have a length of at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 125 nucleotides, at least 150 nucleotides, or at least 180 nucleotides. In some cases, the first nucleotide sequence is generally complementary to a portion of the target nucleic acid. The second nucleotide (i.e., the second hybridizing segment) may also have a length of at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 125 nucleotides, or at least 150 nucleotides (and the length may or may not be equal to the first nucleotide sequence).

A compound probe may include a first oligonucleotide probe comprising a first nucleotide sequence capable of hybridizing to a first target nucleotide sequence in a nucleic acid molecule of interest and a second oligonucleotide probe comprising a second nucleotide sequence capable of hybridizing to a second target nucleotide sequence in the nucleic acid molecule of interest. The degree of hybridization of a nucleotide sequence (e.g., the first nucleotide sequence) to a target nucleotide sequence (e.g., the first target nucleotide sequence) can depend on the particular application and/or hybridization conditions. For instance, in some cases, a nucleotide sequence that hybridizes to a target nucleotide sequence in a nucleic acid molecule of interest may include 100% matched nucleotide pairs (e.g., 100% of the nucleotide sequence of the oligonucleotide probe may hybridize with the target nucleotide sequence). In other cases, a nucleotide sequence that is capable of hybridizing to a target nucleotide sequence may include greater than 95%, greater than 90%, greater than 80%, greater than 70%, greater than 60% matched nucleotide pairs, greater than 40% matched nucleotide pairs, or greater than 20% matched nucleotide pairs. In certain embodiments, the degree of hybridization between a nucleotide sequence (e.g., of an oligonucleotide probe) and a target nucleotide sequence means that these sequences are capable of hybridizing under certain conditions, e.g., under stringent conditions or array assay conditions, i.e., to produce a detectable signal.

A spot of an array can include a homogeneous composition of at least first and second oligonucleotide probes that may be unattached, or attached as a single probe (e.g., a compound probe). In one embodiment, a compound probe includes at least a first oligonucleotide probe comprising a first nucleotide sequence capable of hybridizing to a first target nucleotide sequence in a nucleic acid molecule of interest, and at least a second oligonucleotide probe comprising a second nucleotide sequence capable of hybridizing to a second target nucleotide sequence in the nucleic acid molecule of interest, wherein the first and second nucleotide sequences of the first and second oligonucleotide probes, respectively, may be contiguous with each other on the compound probe or separated from each other by a linker segment on the compound probe, and wherein the first and second nucleotide sequences or first and second nucleotide sequences including the linker segment, together are not genomically contiguous when hybridized to any single strand in the nucleic acid molecule of interest. In certain embodiments, the first and second nucleotides sequences of the first and second oligonucleotide probes, respectively, together are not genomically contiguous when hybridized to any single strand in the nucleic acid molecule of interest, e.g., if the first and second probes are contiguous on the compound probe. In other words, the binding sites for the first and second oligonucleotide probes are not contiguous in a nucleic acid of interest (e.g., a target genome).

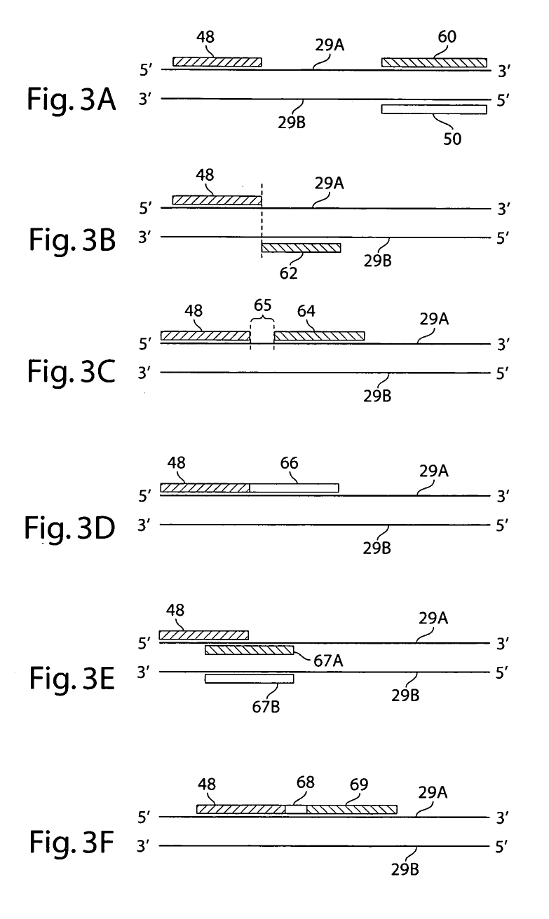

Referring now to both FIGS. 2 and 3, where FIG. 3 illustrates various arrangements of oligonucleotide probes hybridized to target sequences, compound probe 40 of FIG. 2A (and/or compound probes 41 of FIG. 2B) may include probes 48 and 50 that are not genomically contiguous when hybridized to strands 29A or 29B of FIG. 3A. In another embodiment, a compound probe may comprise probes 48 and 60, which are not contiguous on strand 29A of the nucleic acid molecule of interest. In yet another embodiment, as shown in FIG. 3B, a compound probe may include probes 48 and 62 that are also not genomically contiguous on any single strand in the nucleic acid molecule of interest.

In some cases where first and second oligonucleotide probes of a compound probe hybridize to a single strand in the nucleic acid molecule of interest (or hybridize to complementary strands of those regions of interest, this arrangement included as an embodiment), the nucleotide sequences of the first and second probes are separated by a number of bases, for example, at least 1 base, at least 2 bases, at least 5 bases, or at least 10 bases, when hybridized to the single strand. For instance, as shown in FIG. 3C, probes 48 and 64, which may be combined to form a compound probe, may be separated by spacing 65. Spacing 65 may be at least 1 base, at least 2 bases, at least 5 bases, or at least 10 bases long on strand 29A. As shown in FIG. 3D, a probe represented by probes 48 and 66, which are contiguous when hybridized to strand 29A, does not define a compound probe according to some embodiments (e.g., when probes 48 and 66 are contiguous on a single probe), since the individual probes are contiguous when hybridized to the strand.

In some cases, the first and second nucleotide sequences of first and second oligonucleotide probes of a compound probe can overlap if hybridized to a single strand (or a complementary strand) in the nucleic acid molecule of interest. For instance, as shown in FIG. 3E, a compound probe may include probes 48 and 67A, which overlap with each other if each of the probes are hybridized to strand 29A. In another embodiment, a compound probe may include probes 48 and 67B, which overlap if each of the probes are hybridized to complementary strands in the nucleic acid molecule of interest.

In other embodiments, the first and second nucleotide sequences of the first and second oligonucleotide probes of a compound probe, respectively, together can be genomically contiguous when hybridized to any single strand in the nucleic acid molecule of interest, if the first and second sequences of the compound probe are separated by a particular linker segment. For instance, a compound probe can include probes 48 and 66 of FIG. 3D if probes 48 and 66 are not contiguous on the compound probe, e.g., if they are present in compound probe 42 of FIG. 2C as probes 48 and 50. In other embodiments, the first and second nucleotide sequences of the first and second oligonucleotide probes of a compound probe, along with any linker segments that may be present on the compound probe, together are not genomically contiguous when hybridized to any single strand in the nucleic acid molecule of interest. For example, as shown in FIG. 3F, a probe including probes 48, segment 68, and probe 69, in that consecutive order as shown in FIG. 3F (and without any additional linker segments), does not make up a compound probe. However, an embodiment comprising probe 69, segment 68, and probe 48 (e.g., where the 3′ end of probe 69 is connected to the 5′ end of segment 68, and the 3′ end of segment 68 is connected to the 5′ end of probe 48) can comprise a compound probe.

Although the description herein predominately describes probes representing parts of a DNA molecule, it should be understood that probes can represent all, or one or more portions, of other nucleic acid molecules of interest. For example, in some cases, a nucleotide sequence of a compound probe can represent specific parts of a cDNA or a bacterial artificial chromosome (BAC). Probes of a compound probe may be designed to target a genomic region represented by a BAC and the probes may be optimized for stringency, signal to noise, etc. In some embodiments, compound probes are designed to measure a specific genetic marker.

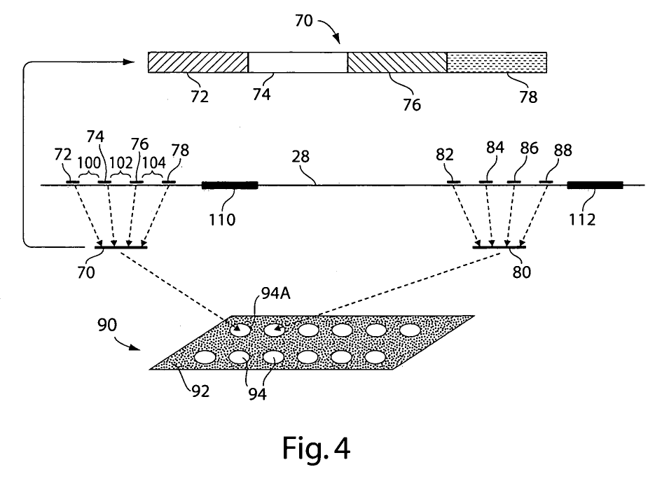

In the embodiment illustrated in FIG. 4, compound probe 70 comprises a series of probes 72, 74, 76, and 78, which can be designed to hybridize to nucleotide sequences located on different parts of a nucleic acid molecule of interest 28. As illustrated in this particular embodiment, the target nucleotide sequences that can hybridize to probes 72, 74, 76, and 78 are not contiguous with each other on the nucleic acid molecule of interest, since they are separated by sections 100, 102, and 104 of the nucleic acid molecule of interest. In one embodiment, sections 100, 102, and 104 each comprise greater than 5 bases. Probes may be separated by a relatively small number of bases (e.g., less than 50 bases) in cases where higher resolution assays are desired. In other cases, sections 100, 102, and 104 may comprise higher numbers of bases (e.g., greater than 100 bases), e.g., when it is desirable to include probes that span nucleic acid molecules of interest having relatively large numbers of bases. As such, the length of sections 100, 102, and 104 can vary depending on the particular application. For example, the average distance between two consecutive probes hybridized to a nucleic acid molecule of interest may be between 0-10 bases, between 1-50 bases, between 50-100 bases, between 100-300 bases, between 300-500 bases, between 500-1000 bases, between 1-10 kb, or greater than 10 kb.

In some cases, the spacing between consecutive probes that are hybridized to a nucleic acid molecule of interest may be substantially equivalent (e.g., consecutive probes may be separated by about 300 bases). In other cases, the spacing between consecutive probes may differ along particular portions of the nucleic acid molecule of interest. For example, if it is known that a biological phenomenon may be associated with a particular portion of the nucleic acid, that portion may include a higher resolution of probes than a portion that is not associated with the biological phenomenon. For example, it is expected that most transcription-factor binding events will occur near the transcription start site of genes.

As shown in FIG. 4, series of probes 72, 74, 76, and 78 that make up compound probe 70 are adjacent to each other along nucleic acid molecule of interest 28, and are genomic neighbors because they are on, or near, one particular gene (e.g., gene 110). A probe that is a genomic neighbor of another probe may be said to be on, or near, the same gene in a nucleic acid molecule of interest. In some cases, the nearness or proximity of a first and a second probe relative to one another may be defined at least in part by a certain number of bases. For instance, a first probe near a second probe may be separated by less than about 10⁷ bases, less than about 10⁶ bases, than about 10⁵ bases, than about 10⁴ bases, less than about 1,000 bases, less than about 500 bases, less than about 300 bases, or less than about 100 bases. In another embodiment, the nearness or proximity of a first and a second probe may be defined at least in part by whether or not they are part of the same gene on the nucleic acid molecule of interest. For example, a first and a second probe that are on or near the same gene may be genomic neighbors and may be said to be near one another, while probes that are on or near different genes in the nucleic acid molecule of interest are not genomic neighbors and are not near one another. In particular embodiment, the binding sites of all of the hybridizing segments of a subject compound probe may be adjacent but not contiguous to each other in a genome.

In other embodiments, a compound probe may include probes that are not on, or near, the same gene in the nucleic acid molecule of interest. E.g., assays may be designed to include compound probes made up of probes that are not located on the same gene in the nucleic acid molecule of interest. For example, in one embodiment, a compound probe may include a first probe on, or near, gene 110 (e.g., one of probes 72, 74, 76, or 78), and a second probe on, or near, gene 112 (e.g., one of probes 82, 84, 86, or 88). In other cases, the compound probe does not include two probes on, or near, gene 110, or two probes on, or near, gene 112. As described in more detail below, such factors are important considerations for designing arrays and for deconvoluting signals obtained from hybridization.

A plurality of compound probes as described herein may be used to form an array. The plurality of compound probes may be present on a surface of the same solid support. The compound probes may be immobilized on, e.g., covalently attached to, different and, in certain aspects, known, locations on the solid support (e.g., substrate surface). In certain embodiments, each distinct compound probe nucleotide sequence of the support is typically present as a composition of multiple copies of the compound probe on the substrate surface, e.g., as a spot or feature on the surface of the substrate. The number of distinct nucleic acid sequences, and hence spots or similar structures, present on the array may vary, but is generally at least 2, usually at least 5 and more usually at least 10, where the number of spots on the array may be as a high as 50, 100, 500, 1000, 10,000 or higher, depending on the intended use of the array. The spots of distinct nucleotide sequences present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. However, in some cases, the distinct nucleotide sequences may be unpatterned or comprise a random pattern.

The density of spots present on the array surface may vary, but will generally be at least about 10 and usually at least about 100 spots/cm², where the density may be as high as 10⁶ or higher, but will generally not exceed about 10⁵ spots/cm². In other embodiments, the polymeric sequences are not arranged in the form of distinct spots, but may be positioned on the surface such that there is substantially no space separating one polymer sequence/spot from another. In one instance, the density of compound probes on the solid support is between about 0.01 and 1 pmol per mm². In other instances, the compound probes may be present on surface at a density of at least about 0.01 pmol/mm², at least about 0.03 pmol/mm², at least about 0.1 pmol/mm², at least about 0.3 pmol/mm², at least about 1 pmol/mm², etc. In one instance, the density of compound probes on the solid support is between about 0.01 pmol/mm² and about 1 pmol/mm².

In some cases, while compound probes at different spots or locations may not be identical, compound probes at a spot are substantially identical, e.g., at least about 25%, at least about 50%, or at least about 75%, or at least about 95% of the compound probes at the feature comprise an identical sequence composition and length. In certain embodiments, each compound probe spot of the array is substantially homogenous or highly uniform in terms of compound probe composition. The length of the compound probes in these cases may be greater than about 60 nucleotides, greater than about 100 nucleotides, greater than about 150 nucleotides, or greater than about 180 nucleotides. Advantageously, background noise and non-selective signal are reduced in the hybridization signal.

As illustrated in the embodiment shown in FIG. 4, compound probes 70 and 80 may be immobilized on, e.g., covalently attached to, locations on solid support 92 (e.g., a substrate surface). Each distinct compound probe (e.g., compound probes 70 and 80) on the support may be present as a homogeneous composition and concentration of multiple copies of the probe on the substrate surface, e.g., as spots 94 on the surface of the substrate.

In one embodiment, arrays of the invention including spots comprising more than one probe on each spot (e.g., compound probes) can be used to determine a location of a biological phenomenon in terms of chromosomal coordinates in a nucleic acid molecule of interest. The array may be contacted with a sample including target nucleotide sequences under conditions that permit hybridization between the target nucleotide sequences and sequences of the oligonucleotide probes on the spots. This may allow hybridization between a target nucleotide sequence of the sample and a sequence of the oligonucleotide probe, and can cause production of a signal on the array as a result of hybridization. In some cases, a signal produced or detected from one spot alone does not enable determination of the particular probe to which the target nucleotide sequence hybridized, nor of where the hybridized target nucleotide sequence is located in the nucleic acid molecule of interest. As such, it may be difficult, or sometimes impossible, to determine the location of a biological phenomenon in terms of chromosomal coordinates from one signal alone. However, in other instances, knowledge of the particular oligonucleotide sequences on a spot, as well as the relationship between where the probes of a spot hybridize in the nucleic acid molecule of interest, can allow determination of some information regarding the location of the biological phenomenon in terms of chromosomal coordinates. In most embodiments, signals from a series of spots are required to give useful information about the location of a biological phenomenon in terms of chromosomal coordinates in the nucleic acid molecule of interest.

Accordingly, it is important in many cases to have at one's disposal a technique for determining the particular probe that contributed to the overall signal of a spot (i.e., a “spot signal”) for arrays including spots comprising at least first and second probes. For instance, if a spot comprised a first probe and a second probe have different nucleotide sequences, a spot signal may indicate hybridization of either the first probe or the second probe (or, in some cases, both probes); however, the spot signal may not (and in many embodiments herein do not) indicate which of the first or second probes gave rise to the signal. In some embodiments, it is not required to determine which particular probe of a spot (e.g., which probe of a compound probe) contributed to the spot signal in order to determine the location of a biological phenomenon in terms of chromosomal coordinates in the nucleic acid molecule of interest. In other embodiments, however, it is necessary to determine which probe on a spot contributed to the overall signal of the spot in order to determine the location of a biological phenomenon in terms of chromosomal coordinates. As will be apparent from the discussion herein, the combination of signals detected from an array or sets of arrays can be used to give useful information such as the particular probe of a spot that gave rise to the spot signal, and/or the general locations of a biological phenomenon in a nucleic acid molecule of interest, and/or specific locations of biological phenomena in terms of chromosomal coordinates in the nucleic acid molecule of interest.

In some instances, arrays or array sets including spots comprising more than one probe (e.g., compound probes) on each spot can be used to decrease the number of arrays necessary to determine one or more locations of a biological phenomenon in terms of chromosomal coordinates in a nucleic molecule of interest, as described in more detail below.

The following discussion will illustrate the advantages of efficiency of array size (or, increase in assay information at a given size), facilitated by the invention. In reference to known arrangements as illustrated in FIG. 1, an assay that queries a biological phenomenon, e.g., the interactions of a transcription factor with the entire human genome, may include the use of probes 10 and 12, where adjacent probes are spaced 300 bases apart. Such an assay may require about 117 arrays, each having 44,000 spots, in order to span the entire genome. For an equivalent assay using the array of FIG. 4 of the present invention, which includes compound probes comprising four different probes (e.g., each probe being a 40 mer) on each spot, and assuming there are 44,000 spots per array, the number of arrays can be decreased. In one embodiment, the number of arrays can be decreased from 117 to 30+1 (the last array being a deconvolution step, described below). The number of arrays can decrease by approximately four-fold, since four times fewer spots are required when using compound probes instead of regular probes. For high density arrays having 95,000, 185,000, or 244,000 features per array, the number of arrays can be decreased from 55, 29, and 22 to 14+1, 8+1, and 6+1 respectively. Using compound probes comprising larger numbers of probes, e.g., 8 probes per compound probe (e.g., each probe being a 20 mer), the number of arrays required can be decreased by about half compared to the assay of FIG. 1. Advantageously, these are significant improvements in the platform as labor and sample costs decrease proportionally with the number of spots and/or assays.

Although the description below focuses primarily on deconvolution of spot signals from spots comprising compound probes, it should be understood that the same techniques are applicable to deconvolution of spot signals from any spot comprising more than one probe. In such embodiments, each probe may be capable of contributing a signal to the spot signal, and the signals from the probes may be indistinguishable from each other. As such, a single spot signal may be an aggregated signal from all of the signals contributed by the probes of the spot.

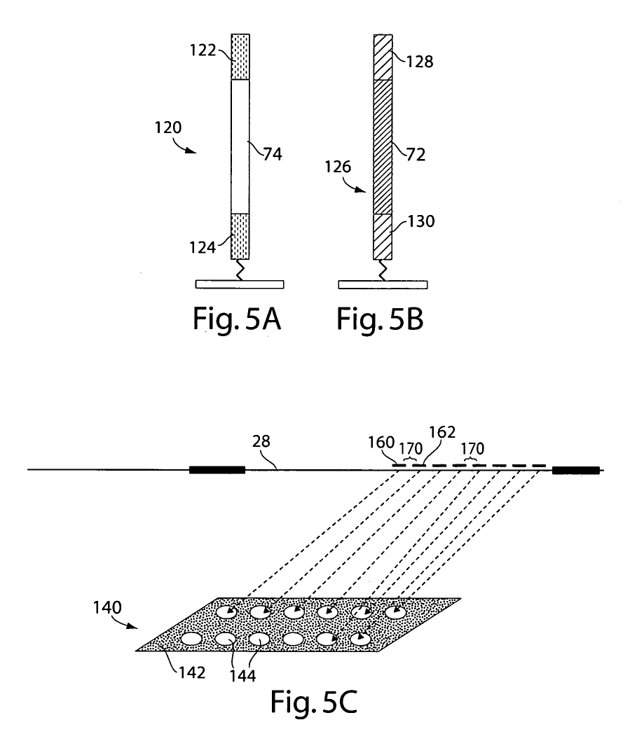

A variety of methods can be used to deconvolute the signals attained from hybridization on an array or on array sets, including signals from spots comprising more than one probe on each spot. In one embodiment, after performing an initial set of assays using array 90, the general areas of interest showing hybridization (i.e., signals or hits) can be deconvoluted by performing a second round of hybridization. This second assay can be designed to tailor the results of the first set of assays and only the hit areas of the first assay can be included. For example, during a first set of assays, if spot 94A of FIG. 4 comprising compound probe 70 produced a signal after hybridization, probes 72, 74, 76 and 78 of compound probe 70 may be included as individual spots in a second assay involving array 140 of FIG. 5C. As shown in the embodiment illustrated in FIG. 5, full length probes can be used in array 140. For instance, probe 74, being a 40 mer in compound probe 70 in FIG. 4, may be included in array 140 as a 60 mer, including regions 122 and 124 that flank probe 74. Regions 122 and 124 may be chosen at least in part by the sequence of the nucleic acid molecule of interest. E.g., when probe 74 is hybridized to the nucleic acid molecule of interest, regions 122 and 124 may also hybridize to the nucleic acid molecule of interest in their positions flanking probe 74. Similarly, probe 72, which was a 40 mer on compound probe 70 of FIG. 4, may be included in array 140 as full length probe 126 including probe 72, as well as regions 128 and 130 that flank probe 72. Regions 128 and 130 may also be chosen at least in part by the sequence of the nucleic acid molecule of interest. Of course, probe 72 may be flanked with only one region 128 or 130. Alternatively, probe 72 may be used as is on array 140, e.g., without flanking regions. The length of probes 120 and 126 can vary, e.g., depending on the assay and/or the hybridization conditions desired. Probes in array 140 may have a length of, for example, greater than 20 nucleotides, greater than 40 nucleotides, greater than 60 nucleotides, greater than 80 nucleotides, or greater than 100 nucleotides.

As illustrated in the embodiment of FIG. 5C, array 140 includes a higher resolution of probes (e.g., a smaller distance between probes on nucleic acid molecule of interest 28) compared to the probes used in array 90 (FIG. 4C). For instance, sections 170 may separate adjacent probes such as probes 160 and 162, and these sections may each comprise fewer numbers of bases that those separating adjacent probes in the first assay involving array 90. E.g., sections 170 may comprise less than about 300 bases, e.g., from about 1-50 bases, from about 50-200 bases, or from about 100-300 bases.

Since spots 144 each comprise a homogenous composition of a single probe, the signals produced or detected after hybridization of the probes and target nucleotides sequences can enable determination of which probe of compound probe 70 gave rise to the spot signal of array 90 of FIG. 4. In some cases, the probes of array 140 can be chosen from the compound probes that gave the strongest signals in array 90, e.g., the probes of the top 10%, top 20%, top 30%, or top 50% of the compound probes that gave the strongest signals may be included in array 140. As such, a single spot of array 140 may allow determination of the location of a biological phenomenon in terms of chromosomal coordinates in the nucleic acid molecule of interest. In some instances, in order to verify a signal from a spot, a series of signals from the spots may be correlated. In other cases, a series of spots may be required to determine the location of a biological phenomenon in terms of chromosomal coordinates in the nucleic acid molecule of interest.

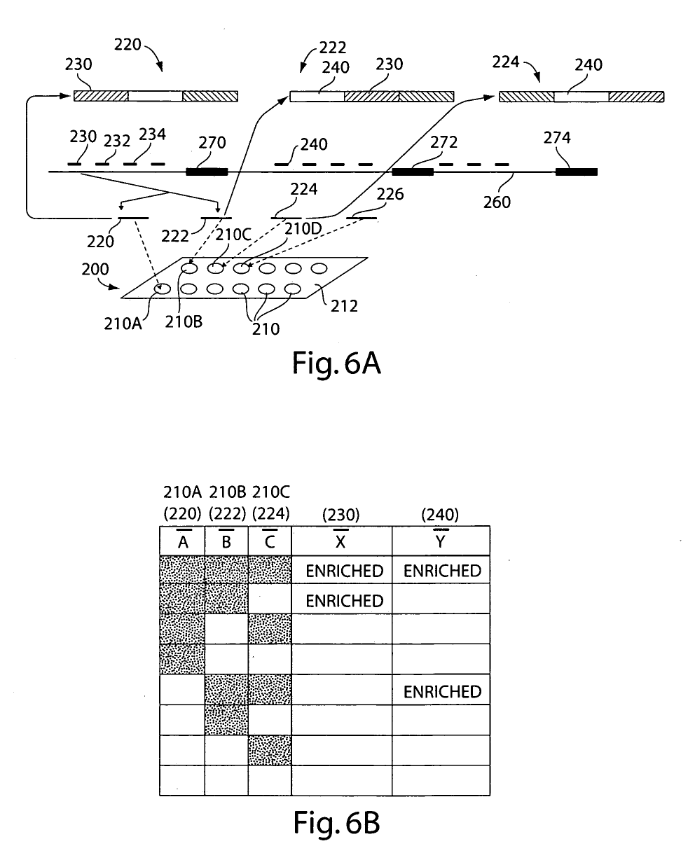

Another assay arrangement and deconvolution technique will now be described. FIG. 6A shows another design of an array including spots comprising a homogenous composition of at least first and second probes according to another embodiment of the invention. Spots 210 of array 200 may include multiple probes in the form of compound probes. For instance, compound probes 220, 222, 224, and 226 may be attached to surface 212 as individual spots 210A, 210B, 210C, and 210D, respectively. In this particular array, the set of compound probes is designed such that every probe of a compound probe is represented multiple times on array 200. For instance, each probe of a compound probe may be present on the array or array set as part of two different compound probes, and on two different spots of the array or array set. In other arrays, compound probes may be represented exactly two times, or exactly three times on an array or array set. Of course, the number of times a probe is represented on an array or array set may vary, e.g., depending on the design of the assay and/or the resolution of the assay. The degree of replication may vary within an array or array set; for example, an array (or array set) may have some probes that are present in two compound probes, some present in three, and some present in four or more.

In the embodiment illustrated in FIG. 6A, the compound probes comprise three probes and each of the probes are represented at least twice as part of different compound probes of the array. The compound probes may be constructed randomly except for the constraint of representing each probe at least twice. In some cases, genomically nearby probes are not included on the same compound probe. For instance, compound probe 220 may include probe 230, which is located on, or near, gene 270 in nucleic acid molecule of interest 260. In one embodiment, the remaining two probes of compound probe 220 are not chosen from the group of probes on, or near, gene 270 (e.g., probes 232 and 234 are not a part of compound probe 220). Probe 230 of compound probe 220 may be represented on a different compound probe of the array; for instance, probe 230 may be present on compound probe 222, which is located on spot 210B of the array. Similarly, compound probe 222 may include 240, which is near gene 272. The remaining probe of compound probe 222 may be chosen from probes close to other genes, such as gene 274. Probe 240 may also be represented twice in the array, e.g., on two different compound probes of the array, such as on compound probe 224 in addition to compound probe 222. In some cases, an array or array set comprises at least two spots comprising a first oligonucleotide probe and at least two spots comprising a second of oligonucleotide probe, wherein the array or array set includes a first spot comprising the first and second oligonucleotide probes (e.g., as a compound probe) and does not include a second spot comprising the first and second oligonucleotide probes. In other cases, the array or array set includes a first spot comprising the first and second oligonucleotide probes and a second spot comprising the first, but not the second oligonucleotide probe. The array or array set can further comprise a third spot that comprises the second oligonucleotide probe but not the first oligonucleotide probe. For example, if a first and a second oligonucleotide probe are included in one spot in the array or array set, then those first and second probes would not normally co-occur on any other spot in the array or array set.

Array 200 of FIG. 6A may be contacted with a sample under conditions that permit hybridization between target nucleotide sequences of the sample and sequences of the oligonucleotide probes. After hybridization and scanning, one or more spots may fluoresce to produce spot signals. In some cases, to determine which probe contributed to the spot signal (e.g., to determine which of the probes of the compound probe the target nucleotide sequence was hybridized), the signal from one spot may be correlated to the signal from another spot. For instance, if spot 210A produced a signal (e.g., a spot signal), it may be useful to look at signals from spots 210B and 210C to determine which probes contributed to the spots signals.

As shown in FIG. 6B, each of spots 210A, 210B, and 210C can each produce signals (illustrated by the shaded areas in FIG. 6B). Since it is known where each probe of a compound probe is located on the array or array set, to determine whether probe 230 contributed to the probe signal of spot 210A (compound probe 220), one can observe whether a similar signal was obtained from spot 210B, which also includes probe 230. In cases when 210A and 210B both produced signals (as in the first two rows of the table), it is likely that 230 contributed to the signals of these spots because both of these spots include probe 230. Similarly, to determine whether probe 240 of spot 210B (compound probe 222) contributed to the spot signal, one can observe whether a similar signal was obtained from spot 210C, which also includes probe 240. If spots 210B and 210C produced signals (as in the first and fifth rows of the table), and both of these spots include probe 240, it is likely that 240 contributed to the signals of these spots. In some cases, a signal of a probe is considered significant if all of the compound probes including that probe sequence show a significant signal. In one particular embodiment, the biological phenomenon is identified if and only if all of the spots comprising probes relating to that phenomenon show a signal. In another embodiment, a significance can be computed for a biological phenomenon at a particular probe based on the significance of the signals of each of the compound probes including that probe, by, for example, computing the joint-likelihood of the pair of signals. Accordingly, enrichment or hybridization of a probe with a target, and the contribution of a signal from one probe among a plurality of probes within a compound probe, can be determined. As such, multiple signals from multiple spots can be correlated to determine the location of a biological phenomenon in terms of chromosomal coordinates in the nucleic acid molecule of interest.

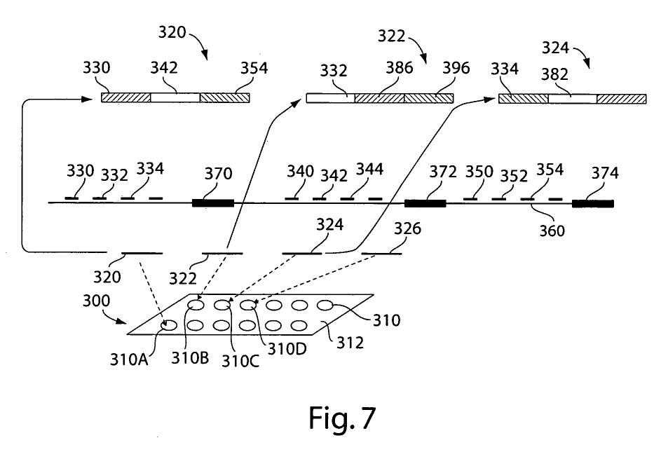

The embodiment illustrated in FIG. 7 shows another arrangement of compound probes on an array, wherein each compound probe comprising three probes. Of course, in other embodiments, each compound probe can comprise any suitable numbers of probes (e.g., four, five, six, or more probes). In one embodiment, each of the probes is represented once in the array (or array set) and probes that are genomic neighbors (or are nearby) are not included on the same compound probe. The compound probes may be constructed randomly, except for these constraints. For instance, compound probe 320 may include probe 330, which is located on, or near, gene 370 in nucleic acid molecule of interest 360. In one embodiment, the remaining two probes of compound probe 320 are chosen randomly, except they are not chosen from the group of probes on, or near, gene 370 (e.g., probes 332 and 334 are not a part of compound probe 320). Instead, the remaining two probes of compound probe 320 may be chosen from the group of probes on, or near, other genes such as gene 372 and/or 374. For example, compound probe 320 may include probe 342, which is on or near gene 372, and probe 354, which is on or near gene 374.

Since in this particular assay, each probe is presented only once, compound probe 322 can have a unique combination of probes compared to compound probe 320. For example, each of the probes of compound probe 322 may be chosen randomly from different portions of nucleic acid molecule of interest 360, each portion being on or near different genes relative to the other portions. Advantageously, such an array can increase the effective resolution of an array by a factor equal to the number of probes in each compound probe. For example, for compound probes having three probes (e.g., as shown in FIG. 7), the effective resolution can increase by a factor of three. Similarly, for compound probes having n probes, the effective resolution can increase by a factor of n.

Arrays (e.g. the array 300 of FIG. 7) may be contacted with a sample under conditions that permit hybridization between target nucleotide sequences of the sample and sequences of the oligonucleotide probes. After hybridization and scanning, one or more spots may fluoresce to produce spot signals. In some cases, it may be desirable to determine which probe contributed to the spot signal (e.g., to determine which of the probes of the compound probe the target nucleotide sequence was hybridized). In other cases, however, it is not necessary to determine which probe contributed to the spot signal in order to determine the location of a biological phenomenon in terms of chromosomal coordinates in the nucleic acid molecule of interest. In some embodiments, the signal from one spot may be correlated to the signal from one or more other spots in order to determine the location of the biological phenomenon.

In one embodiment, the constraints of having probes that are non-genomic neighbors of one another on the same compound probe can aid in the deconvolution of signals obtained upon hybridization. In some cases, knowledge of the expected correlation between neighboring probes can also help in deconvoluting the contribution of each probe of a compound probe from a spot signal.

In some cases, the signal associated with a biological phenomenon at a specific location on a nucleic acid molecule of interest is distributed to probes that are genomic neighbors. For instance, since fragmentation of the nucleic acid of interest is performed randomly, fragments including different nucleotide sequences may include the same signal associated with the biological phenomenon. When the fragment length exceeds the probe spacing (in genomic coordinates), a biological phenomenon can generate a signal that is spread across a set of probes in a genomic region. For example, if the median fragment length is about 800 bp and the average probe spacing is about 30 bp, then a given biological phenomenon can contribute a signal across a genomic “neighborhood” of about 26 probes (e.g., 800 bp divided by 30 bp spacing). Some of the embodiments presented here use this expected correlation among probes that are genomic neighbors for the deconvolution of signals from compound probes.

In one embodiment, processing or deconvolution of signals obtained upon hybridization may be performed at least in part by the fragment distribution, which can be generally approximated (e.g., about 800 bp fragments for a typical ChiP-chip sonication protocol) or inferred (e.g., from precise measurement of individual samples via gel electrophoresis or an Agilent Bio-Analyzer). Deconvolution can be achieved by analyzing a spot signal of compound probes in the genomic context of the probes making up the compound probes. For example, if a particular compound probe including a first and a second probe produces a spot signal, then it can be determined which probe of the compound probe is/are responsible for the signal by looking at the spot signal in the context of the signals of the other compound probes comprising the genomic neighbors of the first probe, and then repeating for the second probe, and so on. The analysis of an expected distribution can take on many forms, e.g., ranging from peak-fitting (e.g., of intensities and/or ratios) to a more comprehensive error model that takes into account the error in the probe intensities and/or knowledge of the expected signal distribution. Such an error model can propagate these errors to make a final estimate of the confidence in identifying signal-producing regions.

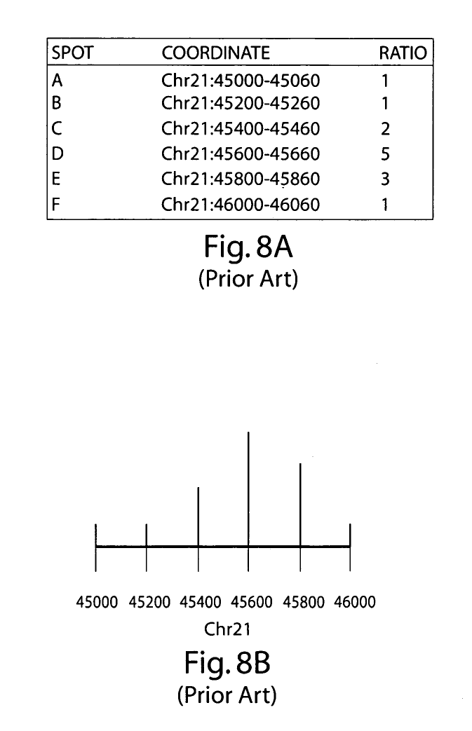

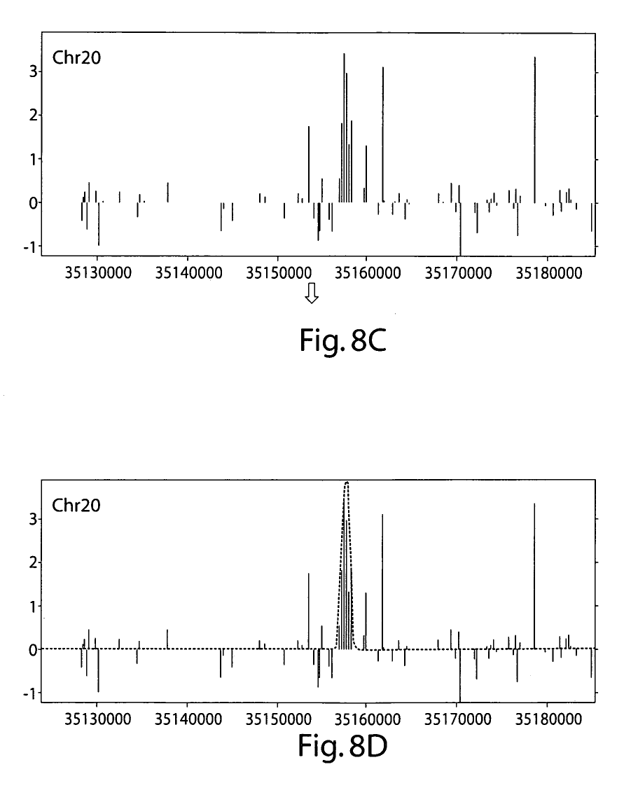

An example of discerning which probe of a compound probe is responsible for a spot signal can be shown in reference to FIG. 7. Since it is known where each probe of a compound probe is located on the array or array set, to determine whether probe 330 contributed to the signal of spot 310A (compound probe 320), one can observe whether signals were obtained from the genomic neighbors of probe 330. For example, signals from spots comprising probes 332 and 334 (e.g., spots 310B and 310C, respectively) may be analyzed together with the signal from spot 310A, because the signal associated with a biological phenomenon at a particular location on nucleic acid of interest 360 may be distributed to probes that are genomic neighbors. In some cases, if the signals arising from probes that are genomic neighbors form an expected distribution of signals (e.g., a Gaussian distribution), the presence of the expected distribution may indicate the location of the biological phenomenon, e.g., at the peak of the distribution. The fitting of shape of the distribution to signals are shown, for example, in FIGS. 8C and 8D. Note that in this example, no fit is found for probes exhibiting high signals inconsistent with neighboring probes. The absence of an expected distribution may indicate the absence of a biological phenomenon at that particular location. Similarly, probe 342 of compound probe 320 may be analyzed in connection with the genomic neighbors of probe 342 (e.g., probes 340 and 344), and the distribution of signals across those genomic neighbors may indicate the presence or absence of a biological phenomenon at that particular location along nucleic acid molecule of interest 360. Accordingly, in one embodiment, the biological phenomenon is identified if and only if all of the spots comprising probes in the genomic neighborhood of the phenomenon show a signal.

FIGS. 8A and 8B show an example of signals that may be generated using an array including typical probes, such as array 30 of FIG. 1C. In array 30, each spot 34, represented as spots A-F in FIG. 8A, can each comprise probes that are 60 bp in length. For example, spot A may include probes that are 60 mers located on chromosome 21 (Chr21) between bases 45,000-45,060. If the probes of spots A and B are separate by 140 bases, and the probes of spot B are also 60 mers, the probes of spot B may be located on Chr21 between bases 45,200-45,260. Since the probes on spots A-F are genomic neighbors and the signal associated with a biological phenomenon at a particular location in the nucleic acid molecule of interest is distributed to probes that are genomic neighbors, the distribution of signals across spots A-F can indicate the presence or absence of a biological phenomenon at that particular location. For instance, the intensities of the signals arising from spots A-F may follow an expected distribution of signals over a genomic region based on fragmentation. Since the distribution of signals shown in FIG. 8B is consistent with the expected distribution, this distribution indicates that a biological phenomenon is located at or near Chr21 base number 45,600.

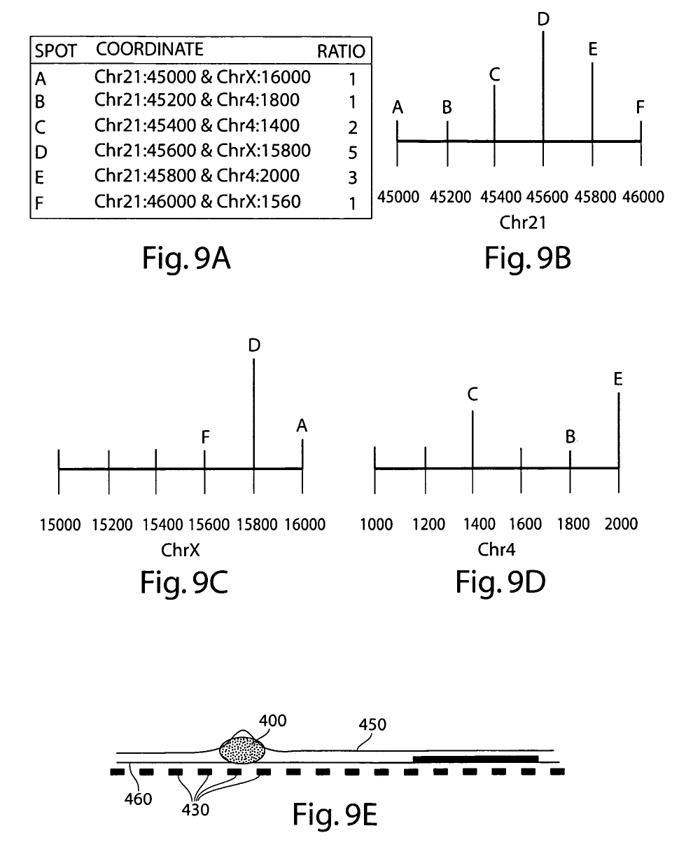

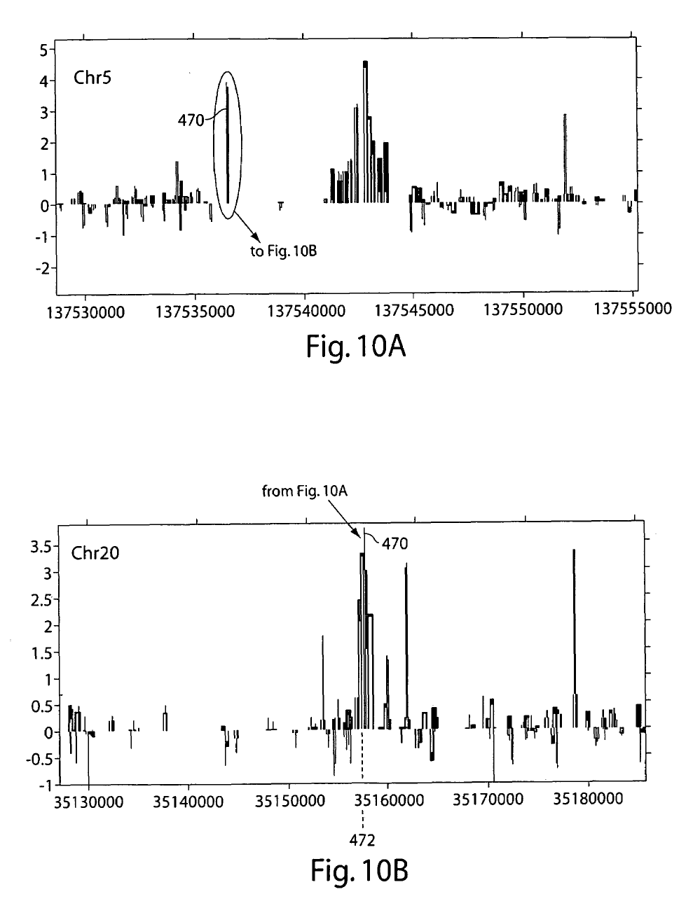

In order to deconvolute signals obtained upon hybridization of compound probes in array 300 of FIG. 7, a similar approach as that described for FIG. 8 is followed. However, because each spot of array 300 comprises multiple probes, additional information can be obtained from each spot, as described below. To simplify the analysis, compound probes including only two probes are described in FIG. 9. The same analysis can be applied to compound probes including three or more probes (e.g., as shown in FIG. 7).

For an array including compound probes comprising first and second probes that are not genomic neighbors on the nucleic acid molecule of interest, each spot generates one spot signal, but this one signal can give useful information about two particular positions on the nucleic acid molecule of interest. (Similarly, a compound probe including three probes can produce one signal that can give useful information about three particular positions on the nucleic acid molecule of interest.) As illustrated in one example shown in FIG. 9A, spot A includes a first probe located on Chr21 at base number 45,000, and a second probe located on chromosome X (ChrX) at base number 16,000 on the nucleic acid molecule of interest. The signal of spot A, which may be shown as a ratio of signals (e.g., a ratio of the spot signal to a base signal), may be plotted along the coordinates of the nucleic acid molecule of interest, e.g., as shown in FIGS. 9B and 9C. Similarly, spot B comprising a first probe located on Chr21 at base number 45,200, and a second probe located on chromosome 4 (Chr4) at base number 1,800 can produce a signal with a ratio of 1 that can be plotted as shown in FIGS. 9B and 9D. A similar approach can be followed for all of the spots of the array, and each signal may be evaluated in connection with the signals from genomic neighbors. In such cases, a signal of a probe may be considered significant if the compound probes which include probes that are genomic neighbors show a significant or expected signal.

As shown in the embodiment illustrated in FIG. 9, spot D produces a signal with a ratio of 5, which may indicate that a biological phenomenon is associated with the probes that make up the compound probe of spot D. In order to determine which probe contributed to the signal at spot D, the signals of the genomic neighbors of the probes of spot D may be analyzed, e.g., as shown in FIGS. 9B and 9C. FIG. 9B shows an expected distribution of signals around Chr21 at base number 45,600, which indicates that the biological phenomenon is likely associated with that position on the nucleic acid molecule of interest. In contrast, the distribution of signals shown in FIG. 9C, is not consistent with an expected distribution, which implies that the biological phenomenon is likely not associated with ChrX at base number 15,800. Advantageously, the signals arising from neighboring probes can be used to differentiate signal (e.g., hybridization on Chr21 at base number 45,600) from noise (e.g., hybridization on ChrX at base number 15,800). FIG. 9E shows the relationship between a biological phenomenon, indicated here by the binding of transcription factor 400 with nucleic acid molecule of interest 460, and probes 430 that give rise to signal 450.

It should be understood that while the description herein involves using separate processing or deconvolution methods for each array or array set, in other embodiments, two or more such techniques can be used in conjunction for a single array or array set. For example, in one embodiment, an array or array set can involve both the use of replicate probes and genomic adjacency. In such instances, the deconvolution methods can depend on both replication (for particular probes that were replicated) and genomic adjacency to determine underlying biological events.

FIG. 10 shows data collected on an array where 120-mer multiplex probes were designed for areas of the genome known to be bound by the transcription factor E2F4. A ChIP-chip assay was performed on HeLa cells using an antibody specific to E2F4, and the resulting amplified and labeled material was hybridized to the array. FIG. 10 illustrates the deconvolution of signals obtained from compound probes of an array. In this particular embodiment, the compound probe comprises a first probe located on chromosome 5 (Chr5) and a second probe located on chromosome 20 (Chr20). After hybridization and scanning, a strong signal 470 was produced (indicated by the height of the bar in the graph). In order to determine which probe contributed to the signal, signal 470 can be correlated with signals, or absence of signals, obtained from its genomic neighbors. For instance, the genomic neighbors of the first probe located on Chr5 of FIG. 10A did not produce an expected distribution of signals, likely indicating that the first probe did not contribute to the signal of the spot. However, second probe located on Chr20 of FIG. 10B, when correlated with the signals from its genomic neighbors, did give a distribution that is consistent with an expected distribution of signals. This indicates that the second probe located at Chr20 gave rise to the signal of the spot. In turn, this also indicates that a biological phenomenon was likely associated with Chr20 at position 472. Advantageously, a single compound probe can provide information that can be associated with multiple chromosomal locations. In addition, the signals, or absence of signals, arising from genomically neighboring probes can be used to differentiate signal from noise. As such, arrays including multiple probes per spot (e.g., compound probes) can be used to decrease the number of arrays necessary to query regions of interest in a biological sample and/or to increase the resolution of biological events.

In one embodiment, additional deconvolution or decoding or signals can be achieved by substituting surrogate base-line measurements for probes at certain locations in cases where high signals are attributed to phenomena at other genomic locations. As described above in connection with FIG. 10, for example, the high enrichment of the probe representing both genomic locations 470 and 472 is attributed to location 472 because its genomic neighbors at that location exhibit the expected distribution. As this attribution is made, the high enrichment at location 470 can be replaced with a base-line value, such as a ratio of one, in order to facilitate further analysis. After the substitution is made, the enrichment at position 470 in FIG. 10A will be low (log-ratio of 0), while the enrichment at position 472 in FIG. 10B will be preserved.

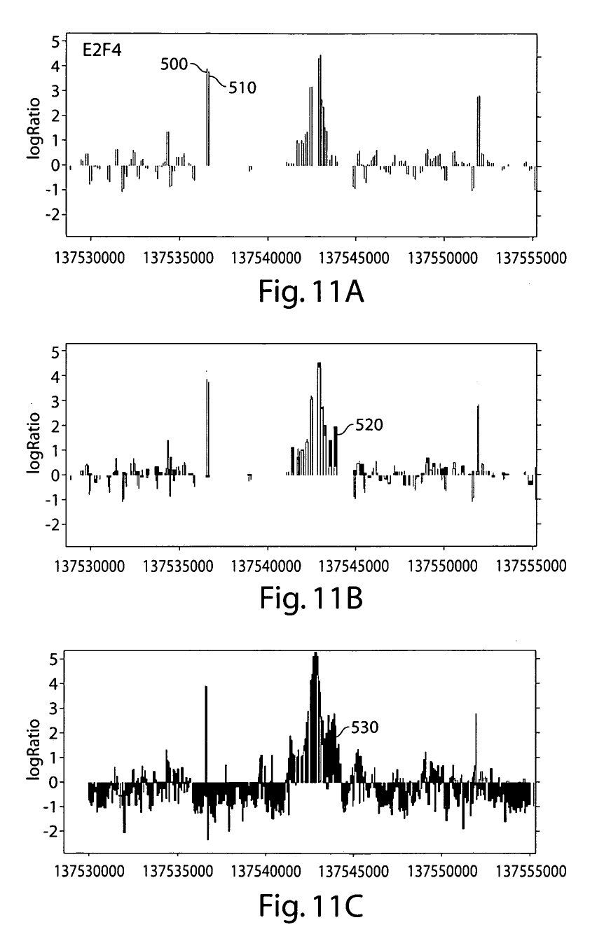

In FIG. 11A, the enrichment is displayed for compound probes assembled from component probes in alternate orderings. The light shaded-regions 500 correspond to probes in a first position of a compound probe, and the dark-shaded regions 510 correspond to probes in a second position of the compound probe. In FIG. 11B, the same data is compared to the signals 520 of conventional (non-compound) probes representing the same genomic locations. In FIG. 11C, the same data is compared to signals 530 of conventional probes representing the same genomic region, but with a higher density of probes.

In addition to the methods described above, methods that increase the ability to resolve underlying biological events as well as overall signal-to-noise performance, through design of the compound probes, are now described. In some embodiments, these methods involve decreasing the amount of homology noise in a compound probe. As used herein, “homology noise” refers to a signal for a probe that arises due to the hybridization of DNA fragments to it that do not correspond to the genomic location it represents. This behavior can occur, for instance, when DNA fragments from different locations in the genome have sequences similar to all, or a portion of, a probe (e.g., high homology). This behavior can also occur in some methods involving formation of compound probes, e.g., when sequences that form the hybridizing segments of the compound probe are concatenated, creating new sequences at the concatenation point.

In one embodiment, a method to reduce boundary homology noise in a compound probe (e.g., reduce the probability of nucleotide sequences that span concatenation points at the probe boundaries being unintentionally homologous with other parts of the genome) includes the use of linker segments between probes. Linker segments, as shown in embodiment 52 of FIG. 2C, may be carefully selected for each adjacent pair of probes within a compound probe to minimize homology noise. For instance, for a compound probe including first and second probes having first and second nucleotide sequences, respectively, a boundary region created by the first and second nucleotide sequences and the linker segment may produces less noise than a boundary region created by the first and second nucleotide sequences without the linker segment, when hybridized to target nucleotides sequences of a biological sample.

Typically, linker segments are short sequences added between two probes of a compound probe. These segments may be, for example, less than 20 bp, less than 10 bp, less than 6 bp, or less than 4 bp in length. However, in other embodiments, longer linker segments may be used. Linker segments may have a variable length, e.g., within a compound probe or between compound probes. In one embodiment, the length and/or sequences of linker segments are randomly selected and/or randomly assigned to compound probes. In another embodiment, the length and/or sequences of linker segments can be selected based on a pre-computed database of linker segments with good homology scores, which indicate low homology noise. For instance, the database of linker segments may be derived at least in part by genomes of other organisms. Or, the database of linker segments may be derived at least in part by sections of the nucleic acid molecule of interest that are known to have good homology scores. For instance, sequences that are known to not show up frequently in the nucleic acid molecule of interest may be suitable linker segments for use in some compound probes.

In certain embodiments, homology noise may be reduced by at least 10%, at least 20%, at least 30%, at least 50%, at least 60%, at least 70%, or at least 90%, using the instant methods.

In one embodiment, a method of assigning at least a first probe and a second probe to a compound probe includes identifying the boundaries between the first and second probes. The amount of homology noise between the probe boundaries (e.g., of the first and second probes) and a particular sequence and a nucleic acid molecule of interest may be analyzed. If the noise between the probe boundaries and sequences of the nucleic acid molecule of interest is low, the linker segment between the first and second probes may not be required. However, if the noise is high, a suitable linker segment may be positioned between and first and second probes in the compound probe. As described above, a database of linker segments may identify the unique sequence that is suitable for the insertion between the first and second probes in order to decrease the amount of homology noise. Of course, the boundary region between first and second probes can differ depending on the order of the first and second probes on the compound probe. For instance, as shown in FIGS. 2A and 2B, the order of probes on a compound probe can differ. As part of the analysis of identifying suitable boundaries between probes of the compound probe, the noise contribution of each arrangement of probes in a compound probe can be evaluated. As such, the arrangement of probes that gives boundary regions having the lowest amount of noise between regions of the nucleic acid molecule of interest may be chosen.

In another embodiment, a method of assigning at least a first probe and a second probe to a compound probe includes choosing probes that have a low probability of self-hybridization, or of forming undesirable secondary structures, to avoid the formation of, for example, hairpins on the spot. However, in certain embodiments, compound probes including probes that can self-hybridized may be useful as controls. In such embodiments, a compound probe may include a first nucleotide sequence and a second nucleotide sequence, wherein the second nucleotide sequence is the complement of the first nucleotide sequence.

In another embodiment, the arrangement (e.g., ordering) of the probes within the compound probe may be selected to minimize boundary homology noise. This can be done by evaluating at least two, several, or all possible arrangements (and/or a subset of possible arrangements) of probes within a compound probe, and selecting the arrangement expected to have the overall lowest boundary homology noise. In addition, this method can be used in conjunction with the linker method presented previously. For instance, in one-embodiment, a method of designing a compound probe comprises selecting candidate probes for a compound probe, the candidate probes comprising at least a first oligonucleotide probe comprising a first nucleotide sequence capable of hybridizing to a first target nucleotide sequence in a nucleic acid molecule of interest, and at least a second oligonucleotide probe comprising a second nucleotide sequence capable of hybridizing to a second target nucleotide sequence in the nucleic acid molecule of interest. The method can involve estimating the boundary homology noise of at least two possible arrangements of the first and second oligonucleotide probes within a compound probe, and selecting the arrangement estimated to have the overall lowest boundary homology noise. In some cases, the boundary homology noise of all possible arrangements of the first and second oligonucleotide probes within a compound probe can be estimated, and the arrangement estimated to have the overall lowest boundary homology noise can be selected.

In another embodiment, a method of designing one or more compound probes and/or arrays or array sets comprising compound probes can include comparing results of a compound probe array to that of a non-compound probe array.

In cases in which compound probes with linker segments are desired, a method of designing a compound probe may further comprise selecting a linker segment from a database of linker segments. The boundary homology noise of at least two possible arrangements (or in some cases, all possible arrangements) of the first and second oligonucleotide probes together with the linker segment within a compound probe may be estimated, and the arrangement estimated to have the overall lowest boundary homology noise can be selected. The database of linker segments can be derived at least in part by sections of the nucleic acid molecule of interest that are known to have good homology scores and/or at least in part by sections of a genome that is different from that of the nucleic acid molecule of interest (e.g., the genome of another organism).

In some embodiments, the methods described above may use a mechanism to evaluate boundary homology noise. This can be done by using existing sequence matching tools such as BLAST, BLAT, and/or MegaBLAST. The system can exclude the expected genome matches from the probes of a compound probe, and use any remaining matches to assess boundary homology noise. However, in some cases, this method could be very computationally expensive, e.g., for large genomes.

A method that can be more efficient (though in some cases, perhaps less precise) may include simply looking for exact matches of some given length (k) created at probe boundary regions (e.g., with or without a linker segment). This can be done by pre-computing a hash/lookup table of all unique k-length segments for a given genome. To evaluate a concatenation point, a k-size window can move one base pair at a time across the boundary point and each sequence may be looked up in the table to estimate homology noise. The overall boundary noise estimate for the compound probe can include a combination of the noise estimates for each boundary within the compound probe.

In another embodiment, ability to resolve underlying biological events can be controlled by taking advantage of information about expected correlation among probes to allocate probes to compound probes. A simple example was described above: for assays (such as ChIP-Chip assays) where the genomic DNA is fragmented, one can expect genomically adjacent probes, sufficiently close together, to show highly correlated signals. In general, a set of probes with expected correlated signals can be spread out among different compound probes, such that there is only one probe of the set in a given compound probe. Other assays may have other correlations which can be leveraged to increase resolving power and/or to control a particular method of deconvoluting signals from hybridization.

For instance, in another embodiment, if it known that a first and second region of a nucleic acid molecule of interest have a high likelihood of being associated with a biological phenomenon, probes within the first and second regions are not put together in a single compound probe. After this constraint, probes that combine to form a compound probe may be chosen from random positions along the nucleic acid molecule of interest. As such, a compound probe may include only one probe representative of a binding site for a biological phenomenon. Consequently, it may be possible to take a description of an assay and put different design parameters to best allocate probes to compound probes and/or compound probes to a particular arrangement on an array in order to tailor the arrangement of probes and compound probes to a particular assay. For example, in an assay intended to identify transcription factor binding locations, it could be assumed that binding events will occur close to transcription start sites. However, since they could occur elsewhere, probes for such an assay can be selected for locations both close to and far from transcription start sites. When assembling these probes into compound probes, it may be desirable to use probes from different distances from transcription start sites to reduce the likelihood of more than one of the probes being associated with a binding event. Other constraints of assigning probes to compound probes and/or the assignment of compounds to particular spots on an array or array set may allow other associations between signals that can used to increase resolution and/or decrease the number of spots per array.

In another embodiment, consideration of the signal intensity of individual probes may be used to constrain the ways in which they are assembled into compound probes. For example, in two-color assays, enrichment of a probe is determined by comparing the signal intensity of its sample channel to its control channel. In a compound probe designed for such an assay, it may be undesirable to pair a probe with high intensity in the control channel with a probe with a low intensity. Should a biological event occur at the location represented by the low-intensity probe, the increased intensity in the sample channel will be larger than that of the control channel of that probe, but perhaps not larger than that of the control channel of the paired high-intensity probe. In such a case, the enrichment information is lost. To mitigate the loss, probes could be assembled into compound probes only with probes of similar control channel intensity. The intensities can be obtained by prediction based on sequence characteristics (e.g. melting temperature and/or uniqueness) or by empirical measurement of the probe behavior in a non-compound-probe context.

Other methods of probe design criteria, e.g., scoring and scaling of nucleotide sequences, are described in U.S. Pat. No. 6,403,314 by Lange, et al., and may be used in combination with the disclosure herein for designing compound probes (e.g., selecting at least first and second probes of a compound probe).

The spots comprising multiple probes per spot (e.g., compound probes) and arrays of the invention find may use in a variety of different applications, including analyte detection applications in which the presence of a particular analyte in a given sample is detected (e.g., qualitatively or quantitatively). Articles and methods of the invention involving spots comprising multiple probes per spot (e.g., compound probes) can be used in any suitable application that uses typical probe arrays such as those shown in FIG. 1. Examples of specific applications include, but are not limited to, array CGH, location analysis (ChIP-Chip), gene synthesis, mutation detection, probe synthesis, aptamer synthesis, therapeutics, microRNA analysis, methylation analysis, amplification methods and the like. Those of ordinary skill in the art may know protocols for carrying out such assays.

Generally, in detection methods relying on oligonucleotides attached to an array, the sample suspected of comprising a target nucleic acid molecule of interest can be contacted with an array under conditions sufficient for the target nucleic acid molecule to hybridize to its respective binding pair member that is present on the array. Thus, if the target nucleic acid molecule of interest is present in the sample, it can hybridize to the array at the site of its binding partner and a complex may be formed on the array surface. The presence of this hybridized complex on the array surface can then be detected, e.g., through use of a signal production system, e.g., an isotopic or fluorescent label present on the target nucleic acid molecule, etc. The presence of the target nucleic acid molecule in the sample can then be deduced from the detection of hybridized complexes on the substrate surface in combination with the methods described herein.

Specific target nucleic acid molecule detection applications of interest include hybridization assays in which the nucleic acid arrays of the present invention are employed. In these assays, a sample of target nucleic acids can first be prepared, where preparation may include labeling of the target nucleic acids with a label, e.g., a member of signal producing system. Following sample preparation, the sample may be contacted with the array under hybridization conditions, whereby complexes can be formed between target nucleic acids that are complementary to probe sequences attached to the array surface. The presence of hybridized complexes can then be detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

In using an array of the present invention, the array will typically be exposed to a sample (for example, a fluorescently labeled target nucleic acid molecule (e.g., protein containing sample)) and the array then read. Reading of the array may be accomplished by illuminating the array and reading the location and intensity of resulting fluorescence at each feature of the array to detect any hybridized complexes on the surface of the array. For example, a scanner may be used for this purpose which is similar to the AGILENT MICROARRAY SCANNER available from Agilent Technologies, Palo Alto, Calif. Other suitable apparatus and methods are described in U.S. Patent Publication No. 2002-0160369 A1, entitled “Reading Multi-Featured Arrays” by Dorsel et al.; and U.S. Pat. No. 6,406,849, entitled “Interrogating Multi-Featured Arrays” by Dorsel et al., which are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels (e.g., two-color or multi-colored channels)) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

Kits for use in analyte detection assays are provided. The subject kits at least include the arrays of the subject invention. The kits may further include one or more additional components necessary for carrying out an target molecule detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing a target nucleic acid molecule, buffers such as hybridization buffers, wash mediums, enzyme substrates, reagents for generating a labeled target sample such as a labeled target nucleic acid sample, antibodies for immunoprecipitating nucleic acid molecules bound by proteins of interest, negative and positive controls and written instructions for using the subject array assay devices for carrying out an array based assay. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM or diskette.

A variety of deconvolution techniques are described herein which may be assisted by computational tools. Techniques are further described in U.S. patent application filed on even date herewith entitled “Analysis of Arrays,” by Gordon, et al. Those of ordinary skill in the art, with the benefit of the present disclosure and knowledge available in the state of the art, can develop and construct necessary algorithms and other software and hardware-associated tools to quickly and efficiently reduce initial assay information to ultimately desired binding information without undue experimentation.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing, ““involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An oligonucleotide probe comprising a plurality of hybridizing segments, wherein said hybridizing segments hybridize to non-contiguous regions in a target genome.

2. The oligonucleotide probe as in claim 1, wherein said oligonucleotide probe is at least 100 bases in length.

3. The oligonucleotide probe as in claim 1, wherein said hybridizing segments are each at least 40 bases in length.

4. The oligonucleotide probe as in claim 1, wherein said oligonucleotide probe comprises at least 3 hybridizing segments.

5. The oligonucleotide probe as in claim 1, wherein said hybridizing segments are contiguous.

6. The oligonucleotide probe as in claim 1, wherein said hybridizing segments are separated by a linker.

7. The oligonucleotide probe as in claim 1, wherein said linker comprises a nucleotide sequence that is optimized to minimize hybridization to the linker.

8. The oligonucleotide probe as in claim 1, wherein said non-contiguous regions are separated by at least 10 kb in a target mammalian genome.

9. The oligonucleotide probe as in claim 8, wherein said non-contiguous regions are present on different chromosomes of said mammalian genome.

10. An array comprising an oligonucleotide probe of claim 1.

11. A method of sample analysis:
a) contacting a sample comprising nucleic acids with an array of claim 10 under hybridizing conditions; and
b) assessing binding of said nucleic acids with said oligonucleotide.

12. A kit comprising the array of claim 10.

13. A compound probe, comprising:
at least a first oligonucleotide probe comprising a first nucleotide sequence capable of hybridizing to a first target nucleotide sequence in a nucleic acid molecule of interest; and
at least a second oligonucleotide probe comprising a second nucleotide sequence capable of hybridizing to a second target nucleotide sequence in the nucleic acid molecule of interest,
wherein the first and second nucleotide sequences of the first and second oligonucleotide probes, respectively, together are not genomically contiguous when hybridized to any single strand in the nucleic acid molecule of interest.

14. The compound probe as in claim 13, wherein the first and second nucleotide sequences of the first and second oligonucleotide probes, respectively, are contiguous with each other on the compound probe.

15. The compound probe as in claim 13, wherein the first and second nucleotide sequences of the first and second oligonucleotide probes, respectively, are separated from each other by a linker segment on the compound probe, and wherein the first and second nucleotide sequences including the linker segment, together are not genomically contiguous when hybridized to any single strand in the nucleic acid molecule of interest.

16. An array or array set for determining a location of a biological phenomenon in terms of chromosomal coordinates in a nucleic acid molecule of interest, comprising:
a plurality of spots, each spot comprising a homogenous composition of nucleotide sequences, each composition of a spot comprising:
at least a first oligonucleotide probe comprising a first nucleotide sequence capable of hybridizing to a first target nucleotide sequence in a nucleic acid molecule of interest; and
at least a second oligonucleotide probe comprising a second nucleotide sequence capable of hybridizing to a second target nucleotide sequence in the nucleic acid molecule of interest,
wherein the first and second nucleotide sequences of the first and second oligonucleotide probes, respectively, may be contiguous with each other on the compound probe or separated from each other by a linker segment on the compound probe, and wherein the first and second nucleotide sequences or first and second nucleotide sequences including the linker segment, together are not genomically contiguous when hybridized to any single strand in the nucleic acid molecule of interest.

17. A kit for determining a location of a biological phenomenon in terms of chromosomal coordinates in a nucleic acid molecule of interest, comprising:
an array or array set comprising a plurality of spots, each spot comprising a homogenous composition of nucleotide sequences, each composition of a spot comprising:
at least a first oligonucleotide probe comprising a first nucleotide sequence capable of hybridizing to a first target nucleotide sequence in a nucleic acid molecule of interest; and
at least a second oligonucleotide probe comprising a second nucleotide sequence capable of hybridizing to a second target nucleotide sequence in the nucleic acid molecule of interest,
wherein the first and second nucleotide sequences of the first and second oligonucleotide probes, respectively, may be contiguous with each other on the compound probe or separated from each other by a linker segment on the compound probe, and wherein the first and second nucleotide sequences or first and second nucleotide sequences including the linker segment, together are not genomically contiguous when hybridized to any single strand in the nucleic acid molecule of interest.

18. A method of determining a location of a biological phenomenon in terms of chromosomal coordinates in a nucleic acid molecule of interest, comprising:
contacting a sample comprising including target nucleotide sequences with an array comprising a plurality of oligonucleotide probes under conditions that permit hybridization between target nucleotide sequences of the sample and sequences of the oligonucleotide probes, and allowing hybridization of a target nucleotide sequence of the sample and a sequence of the oligonucleotide probe;
detecting a signal on the array or array set as a result of hybridization;
correlating the signal to at least two locations on the nucleic acid molecule of interest; and
determining a location of a biological phenomenon in the nucleic acid molecule of interest.

19. A method of determining a location of a biological phenomenon in terms of chromosomal coordinates in a nucleic acid molecule of interest, comprising:
contacting a sample comprising target nucleotide sequences with an array or array set comprising a plurality of oligonucleotide probes under conditions that permit hybridization between target nucleotide sequences of the sample and sequences of the oligonucleotide probes, and allowing hybridization of a target nucleotide sequence of the sample and a sequence of the oligonucleotide probe;
producing a signal on the array or array set as a result of hybridization, wherein the signal of a spot alone does not enable determination of the target nucleotide sequence hybridized on the spot;
detecting hybridization; and
determining a location of a biological phenomenon in the nucleic acid molecule of interest using a combination of signals produced after hybridization.

20. A method of assaying nucleotide sequences in a biological sample, comprising:
contacting a biological sample including target nucleotide sequences with the plurality of compound probes of the first array or array set comprising:
a plurality of compound probes, each compound probe comprising at least a first probe including a first nucleotide sequence complementary to a first nucleotide sequence in a biological sample and at least a second probe comprising a second nucleotide sequence complementary to a second nucleotide sequence in the biological sample;
under conditions that permit hybridization of complementary sequences between the target nucleotide sequences of the biological sample and nucleotide sequences of the first array or array set;
detecting hybridized compound probes of the first array or array set, wherein the hybridized compound probes can be hybridized partially or completely;
contacting the biological sample including target nucleotide sequences to the plurality of probes of the second array or array set comprising:
a plurality of probes including the first and second probes of the hybridized compound probes of the first array or array set
under conditions that permit hybridization of complementary sequences between the target nucleotide sequences of the biological sample and nucleotide sequences of the second array or array set; and
detecting hybridized probes of the second array or array set.
